(19) United States
(12) Patent Application Publication
(10) Pub. No.: US 2025/0000648 A1
(43) Pub. Date: Jan. 2, 2025

(54) DELIVERY APPARATUS FOR A MECHANICALLY EXPANDABLE PROSTHETIC HEART VALVE

(71) Applicant: EDWARDS LIFESCIENCES CORPORATION, Irvine, CA (US)

(72) Inventors: Noam Miller, Givatayim (IL); Assaf Sharon, Tel Aviv (IL)

(21) Appl. No.: 18/883,921

(22) Filed: Sep. 12, 2024

Related U.S. Application Data
(63) Continuation of application No. PCT/US2023/015070, filed on Mar. 13, 2023.
(60) Provisional application No. 63/325,582, filed on Mar. 30, 2022.
(60) Provisional application No. 63/319,702, filed on Mar. 14, 2022.

(51) Int. Cl. A61F 2/24

(52) U.S. Cl. CPC A61F 2/243; A61F 2/2418

(57) ABSTRACT

A prosthetic device, such as a prosthetic heart valve, can include a frame comprising a plurality of axially extending posts and a plurality of struts that connect the plurality of axially extending posts. One or more of these posts can be configured as expansion and locking mechanisms. For example, the posts that are configured as expansion and locking mechanisms can include proximal and distal members that are axially movable relative to one another, and are moveable via rotation of a threaded rod connected therebetween. An actuation assembly can include three-subassemblies configured for actuation of the threaded rod, and can be configured to limit unwanted axial and rotational movement of the prosthetic heart valve during radial expansion and/or compression of the frame.

### CROSS-REFERENCE TO RELATED APPLICATIONS

This application is a continuation of PCT Application No. PCT/US2023/015070, filed Mar. 13, 2023, which claims the benefit of U.S. Provisional Patent Application No. 63/319,702, filed Mar. 14, 2022, and U.S. Provisional Patent Application No. 63/325,582, filed Mar. 30, 2022, each of which is incorporated by reference herein in its entirety.

### FIELD

The present disclosure relates to apparatus and methods for delivering, expanding, implanting, and deploying implantable, radially expandable prosthetic devices, such as mechanically expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various fully functional working diameters. Some mechanically expandable prosthetic heart valves can also be compressed after an initial expansion (for example, for repositioning and/or retrieval).

Despite the recent advancements in percutaneous valve technology, there remains a need for improved mechanically expandable transcatheter prosthetic devices and transcatheter deployment or actuator mechanisms that control radial expansion and retraction of the expandable prosthetic devices. For example, using a conventional actuator for radial expansion of a mechanically expandable prosthetic heart valve can cause unwanted movement and/or rotation of the prosthetic valve, which may result in the prosthetic valve being mispositioned within the diseased native valve. As another example, detachment of conventional actuators from a frame of the prosthetic valve after actuation may be difficult and/or cause unwanted movement and/or rotation thereof.

### SUMMARY

Described herein are delivery apparatus for prosthetic heart valves, and methods for implanting prosthetic heart valves utilizing a delivery apparatus, as well as methods for assembly of a delivery apparatus. The disclosed delivery apparatus and methods can, for example, limit unwanted axial and rotational movement of a prosthetic heart valve during radial expansion or compression of a frame of the prosthetic heart valve via an actuation assembly of the delivery apparatus, and/or during separation of the actuation assembly from the prosthetic valve after radial expansion or compression of the frame. As such, the devices and methods disclosed herein can, among other things, overcome one or more of the deficiencies of typical delivery apparatus and methods of prosthetic heart valve implantation.

A delivery apparatus for a prosthetic implant can comprise a handle and one or more shafts coupled to the handle. In addition, a delivery apparatus can comprise an actuation assembly configured to mechanically expand a frame of a prosthetic heart valve. Further, a delivery apparatus can further comprise one or more of the components disclosed herein.

In some examples, an actuation assembly can include a first member comprising threads disposed on a distal end portion thereof that are configured to form a first releasable connection with a threaded portion of the head of the actuator member of the frame.

In some examples, an actuation assembly can include a second member that is coaxial with the first member and has distal end portion configured to form a second releasable connection with the head of the actuator member.

In some examples, the distal end portion of the second member has a shape that is configured to mate with and fit within or around a complementary-shaped portion of the head of the actuator member such that the second releasable connection is formed.

In some examples, the actuation assembly can include a third member that is coaxial with and disposed around each of the first member and the second member, wherein the first member and the second member are rotatable relative to the third member.

In some examples, the first member can extend coaxially through the second member.

In some examples, the threads of the first member can be external threads that are configured to mate with internal threads of a first socket of the head of the actuator member.

In some examples, the distal end portion of the second member comprises a second socket defining an interior surface comprising one or more first facets, the one or more first facets having a complementary configuration to one or more second facets on an exterior surface of the head of the actuator member.

In some examples, the second member can extend coaxially through the first member.

In some examples, the threads of the first member can be internal threads disposed on a socket of the of the distal end portion of the first member that are configured to mate with external threads on a threaded portion of the head of the actuator member.

In some examples, the distal end portion of the second member can be an actuation head having a cross-section shaped to fit within a complementary shaped socket of the head of the actuator member.

In some examples, an actuation assembly can include an outer sleeve sub-assembly comprising a sleeve head configured to engage opposing surfaces of an end portion of the frame.

In some examples, an actuation assembly can include an actuation sub-assembly extending through the outer sleeve sub-assembly, the actuation sub-assembly comprising an actuation head comprising one or more planar surfaces configured to engage one or more complementary planar surfaces of the head of the actuator member.

In some examples, an actuation assembly can include an engagement sub-assembly extending through the outer sleeve sub-assembly, the engagement sub-assembly comprising an engagement head comprising a threaded portion configured to form a releasable threaded connection with a threaded portion of the head of the actuator member.

In some examples, the engagement sub-assembly extends coaxially through the actuation sub-assembly such that at least a portion of the engagement head extends into at least a portion of the actuation head, the engagement head rotatable relative to the actuation head.

In some examples, the actuation sub-assembly can extend coaxially through the engagement sub-assembly such that at least a portion of the actuation head extends into at least a portion of the engagement head, the engagement head rotatable relative to the actuation head.

In some examples, an actuation assembly can include an outer sleeve member and a sleeve head disposed at a distal end of the outer sleeve member and comprising a first support extension and a second support extension.

In some examples, the actuation assembly can be configured such that, in the engaged position, the first support extension and the second support extension engage opposing exterior and interior surfaces, respectively, of an end portion of the frame.

In some examples, an actuation assembly can be configured such that, in an engaged position with the frame, the sleeve head and the sleeve member are non-rotatable relative to the frame.

In some examples, an actuation assembly can include an actuation member coaxially disposed within a lumen of the outer sleeve member which is rotatable relative to the outer sleeve member, and an actuation head disposed at a distal end of the actuation member.

In some examples, an actuation head can include a first socket, at least a portion of the actuation head extending through at least a portion of a channel within a sleeve head, the actuation head rotatable relative to the sleeve head.

In some examples, the first socket can include one or more facets for engagement with a faceted head of an actuator member of the frame.

In some examples, an actuation assembly can include an engagement member coaxially disposed within a lumen of the actuation member, the engagement member rotatable relative to each of the sleeve member and the actuation member, and an engagement head disposed at a distal end of the engagement member, at least a portion of the engagement head disposed within the actuation head, the engagement head being rotatable relative to each of the sleeve head and the actuation head.

In some examples, the engagement head can be a threaded member that can engage a second, threaded socket within the head of the actuation member of the frame.

In some examples, the engagement head is configured to retain engagement between the actuation assembly and the head of the actuation member of the frame, while the actuation head and the engagement head are rotated to actuate the actuation member of the frame.

In some examples, an actuation assembly can include a first member comprising a threaded socket at its distal end that is configured to engage with external threads on a threaded portion of a head of an actuator member of the frame.

In some examples, an actuation assembly can include a second member disposed coaxially within the first member, the second member comprising an actuation head at its distal end that is shaped to mate with and fit within a socket of the head of the actuator member of the frame, wherein the first member and the second member are rotatable together to rotate the actuator member and expand the prosthetic valve.

In some examples, in an engaged position, the threaded socket of the first member can be engaged with the external threads on the threaded portion of the head of the actuator member and the actuation head of the second member can be disposed within and engaged with the socket of the head of the actuator member, and the first member and the second member can be rotatable together to rotate the actuator member and radially expand or compress the frame.

In some examples, an actuation head of the actuation assembly has a cross-sectional shape that is complementary to a cross-sectional shape of the socket of the head of the actuator member.

In some examples, a system can include a prosthetic valve and a delivery apparatus including an actuation assembly.

In some examples, the prosthetic valve can include a mechanically actuatable frame, the mechanically actuatable frame comprising at least one actuator member, the actuator member configured to be rotated, via rotation of a head thereof, to radially expand or compress the frame.

In some examples, the actuation assembly of the delivery apparatus can include an outer sleeve member, an actuation member, and an engagement member.

In some examples, the sleeve member can include a stationary coupler disposed at a distal end thereof and configured to releasably couple with an end portion of the frame.

In some examples, the actuation member can include an actuation head disposed at a distal end thereof and comprising a first rotatable coupler configured to releasably couple with a first portion of the head of the actuator member.

In some examples, the engagement member can include an engagement head disposed at a distal end thereof and comprising a second rotatable coupler configured to releasably couple with a second portion of the head of the actuator member.

In some examples, the stationary coupler can include a pair distally extending support extensions configured to be slid over an interior surface and an exterior surface of the end portion of the frame.

In some examples, a distal end of the actuation head and a distal end of the engagement head can be distal relative to distal ends of the pair of distally extending support extensions, and the end portion of the frame can include a pair of arms having the head of the actuator member disposed therebetween, the head being recessed relative to a proximal end of the end portion of the frame.

In some examples, a distal end of the actuation head and a distal end of the engagement head can be proximal relative to distal ends of the pair of support extensions, and the head of the actuator member can extend outwardly from a proximal face of the end portion of the frame.

In some examples, the sleeve head can include a window in a side surface thereof, the window configured to enable visualization of contact between the actuation head and the head of the actuator member.

In some examples, a method can include utilizing a delivery apparatus including an actuation assembly to expand or compress a prosthetic valve.

In some examples, a method can include engaging a sleeve head of an outer sleeve member of the actuation assembly with an end portion of a frame of a prosthetic valve.

In some examples, engaging the sleeve head with the end portion of the frame can include bracing the end portion between first and second support extensions of the sleeve head.

In some examples, a method can include engaging an engagement head of an engagement member of the actuation assembly with a first portion of a head of an actuator of the frame.

In some examples, engaging the engagement head with the first portion of the head of the actuator can include threadedly engaging the engagement head and the first portion of the head of the actuator.

In some examples, a method can include engaging an actuation head of an actuation member of the actuation assembly with a second portion of a head of an actuator of the frame.

In some examples, engaging the actuation head with the second portion of the head of the actuator can include engaging one or more facets or planar surfaces of the actuation head with one or more complementary facets or planar surfaces of the second portion of the head of the actuator.

In some examples, the actuation member coaxially extends through a lumen of the outer sleeve member, and the engagement member coaxially extends through a lumen of the actuation member.

In some examples, the engagement member coaxially extends through a lumen of the outer sleeve member, and the actuation member coaxially extends through a lumen of the engagement member.

In some examples, a method can include rotating the actuation member and the engagement member in a first direction to radially expand the frame.

In some examples, a method can include rotating the actuation member and the engagement member in a second opposing direction to radially compress the frame.

In some examples, a delivery apparatus comprises one or more of the components recited in Examples 1-128 below.

In one representative example, an actuation assembly configured for actuation of a prosthetic valve is disclosed. The actuation assembly comprises a mechanically actuatable frame comprising at least one actuator member, the actuator member configured to be rotated, via rotation of a head thereof, to radially expand or compress the frame, the actuation assembly comprising: an outer sleeve member; a sleeve head disposed at a distal end of the outer sleeve member and comprising a first support extension and a second support extension; an actuation member coaxially disposed within a lumen of the outer sleeve member, the actuation member being rotatable relative to the sleeve member; an actuation head disposed at a distal end of the actuation member and comprising a first socket, at least a portion of the actuation head extending through at least a portion of a channel within the sleeve head, the actuation head being rotatable relative to the sleeve head; an engagement member coaxially disposed within a lumen of the actuation member, the engagement member being rotatable relative to each of the sleeve member and the actuation member; and an engagement head disposed at a distal end of the engagement member, at least a portion of the engagement head disposed within the first socket, the engagement head being rotatable relative to each of the sleeve head and the actuation head; wherein the actuation assembly is configured to be transitioned between an engaged position and a disengaged position with the frame.

In another representative example, an actuation assembly configured for actuation of a prosthetic valve is disclosed. The actuation assembly comprises a mechanically actuatable frame, the mechanically actuatable frame comprising a proximal post, a distal post, and an actuator member, the actuator member configured to be rotated, via rotation of a head thereof, to adjust a distance between the proximal post and the distal post, the actuation assembly comprising: an outer sleeve member; a sleeve head disposed at a distal end of the outer sleeve member and comprising a stationary coupler configured to releasably couple with a portion of the proximal post; an actuation member coaxially disposed within a lumen of the outer sleeve member, the actuation member being rotatable relative to the sleeve member; an actuation head disposed at a distal end of the actuation member and comprising a first rotatable coupler configured to releasably couple with a first portion of the head of the actuator member, the actuation head being rotatable relative to the sleeve head; an engagement member coaxially disposed within a lumen of the actuation member, the engagement member being rotatable relative to each of the sleeve member and the actuation member; and an engagement head disposed at a distal end of the engagement member and comprising a second rotatable coupler configured to releasably couple with a second portion of the head of the actuator member, the engagement head being rotatable relative to each of the sleeve head and the actuation head.

In another representative example, a system is disclosed. The system comprises: a prosthetic valve comprising a mechanically actuatable frame, the mechanically actuatable frame comprising at least one actuator member, the actuator member configured to be rotated, via rotation of a head thereof, to radially expand or compress the frame; and a delivery apparatus comprising at least one actuation assembly, the actuation assembly comprising: an outer sleeve member; a sleeve head disposed at a distal end of the outer sleeve member and comprising a stationary coupler configured to releasably couple with an end portion of the frame; an actuation member coaxially disposed within a lumen of the outer sleeve member, the actuation member being rotatable relative to the sleeve member; an actuation head disposed at a distal end of the actuation member and comprising a first rotatable coupler configured to releasably couple with a first portion of the head of the actuator member, the actuation head being rotatable relative to the sleeve head; an engagement member coaxially disposed within a lumen of the actuation member, the engagement member being rotatable relative to each of the sleeve member and the actuation member; and an engagement head disposed at a distal end of the engagement member and comprising a second rotatable coupler configured to releasably couple with a second portion of the head of the actuator member, the engagement head being rotatable relative to each of the sleeve head and the actuation head.

In another representative example, a method of using an actuation assembly of a delivery apparatus to actuate a mechanically actuatable frame of a prosthetic valve is disclosed. The actuation assembly comprising an elongate member and a head portion, the elongate member comprising a sleeve member, an actuation member coaxially disposed within and rotatably within a lumen of the sleeve member, and an engagement member disposed and being rotatable within a lumen of the actuation member, the head portion comprising a sleeve head coupled to the sleeve member, an actuation head coupled to the actuation member and at least partially disposed and being rotatable within the sleeve head, and an engagement head coupled to the engagement member and at least partially disposed and being rotatable within the actuator head, the mechanically actuatable frame comprising at least one actuator member, the actuator member configured to be rotated, via rotation of a head thereof, to radially expand or compress the frame. The method comprises: aligning the head portion of the actuation assembly with an end portion of the frame; and rotating the engagement member in a first direction to cause a threaded member of the engagement head to be threadedly engaged into a threaded socket within the head of actuator member, the threaded engagement resulting in: axial movement of the engagement head, the actuation head, and the sleeve head in a proximal to distal direction; engagement of a faceted socket of the actuation head with a faceted exterior surface of the head of the actuator member; and engagement of a pair of support extensions of the sleeve head with opposing surfaces of the end portion of the frame.

In another representative example, a delivery apparatus for implanting a prosthetic valve is disclosed. The prosthetic valve comprises a mechanically actuatable frame, the frame comprising at least one actuator member configured to be rotated, via rotation of a head thereof, to radially expand and compress the frame, and the delivery apparatus comprising: at least one actuation assembly configured to form a releasable connection the frame, the actuation assembly comprising: an outer sleeve sub-assembly comprising a sleeve head configured to engage opposing surfaces of an end portion of the frame; an actuation sub-assembly extending through the outer sleeve sub-assembly, the actuation sub-assembly comprising an actuation head configured to engage an exterior surface of the head of the actuator member such that rotation of the actuation head produces rotation of the actuator member; and an engagement sub-assembly extending through the actuator sub-assembly, the engagement sub-assembly comprising an engagement head comprising a threaded portion configured to form a releasable threaded connection with a threaded socket of the head of the actuator member.

In another representative example, an actuation assembly is disclosed. The actuation assembly is configured for actuation of a prosthetic valve comprising a mechanically actuatable frame comprising at least one actuator member, the actuator member configured to be rotated, via rotation of a head thereof, to radially expand or compress the frame, the actuation assembly comprising: an actuation member; an actuation head disposed at a distal end of the actuation member and comprising a first socket configured to releasably couple with a first portion of the head of the actuator member; an engagement member coaxially disposed within a lumen of the actuation member, the engagement member being rotatable relative to the actuation member; and an engagement head disposed at a distal end of the engagement member, at least a portion of the engagement head disposed within the first socket, the engagement head being rotatable relative to the actuation head and configured to releasably coupled with a second portion of the head of the actuator member; wherein the actuation assembly is configured to be transitioned between an engaged position and a disengaged position with the frame.

In another representative example, a method of assembling a delivery apparatus configured for delivery and actuation of a prosthetic valve comprising a mechanically actuatable frame is disclosed. The method comprises: assembling at least one actuation assembly, the assembling comprising: disposing an engagement member coaxially within a lumen of an actuation member, the engagement member rotatable relative the actuation member; disposing an engagement head comprising a threaded member at least partially within a faceted socket of an actuation head, wherein the engagement head is attached at a distal end of the engagement member and the actuation head is attached at a distal end of the actuation member, wherein the engagement head is rotatable relative to the actuation head, and wherein the threaded member is configured for threaded engagement with a threaded socket in a head of an actuation member of the frame, and the faceted socket is configured to engage one or more facets on an exterior of the head of the actuation member; coupling the engagement member of an actuation assembly to a first control mechanism in a handle of the delivery apparatus, the first control mechanism configured to enable rotation of the engagement member in a first direction of the engagement member for threadedly engaging the threaded member into the threaded socket, and further configured to enable rotation of the engagement member in a second direction of the engagement member for threadedly disengaging the threaded member from the threaded socket; and coupling the actuation member of the actuation assembly to a second control mechanism in the handle, the second control mechanism configured to enable rotation of the actuation member in a first direction of the actuation member to transmit torque to the head of the actuation member for radial expansion of the frame, and further configured to enable rotation of the actuation member in a second direction of the actuation member to transmit torque to the head of the actuation member for radial collapse of the frame.

In another representative example, an actuation assembly configured for actuation of a prosthetic valve is disclosed. The prosthetic valve comprises a mechanically actuatable frame comprising at least one actuator member, the actuator member configured to be rotated, via rotation of a head thereof, to radially expand or compress the frame, and the actuation assembly comprises: an outer sleeve sub-assembly comprising a sleeve head having a pair of arms configured to engage opposing surfaces of an end portion of the frame; an actuation sub-assembly extending coaxially through the outer sleeve sub-assembly, the actuation sub-assembly comprising a faceted actuation head configured to engage one or more facets on the head of the actuator member such that rotation of the actuation head produces rotation of the actuator member; and an engagement sub-assembly extending coaxially through the outer sleeve sub-assembly, the engagement sub-assembly comprising an engagement head comprising a threaded portion configured to form a releasable threaded connection with a complementary threaded portion of the head of the actuator member; wherein the actuation assembly is configured to be transitioned between an engaged position and a disengaged position with the frame.

In another representative example, a prosthetic valve configured to be transitioned between a radially compressed state and a radially expanded state via an actuation assembly is disclosed. The actuation assembly comprising an outer sleeve sub-assembly, an actuation sub-assembly coaxially extended through the outer sleeve sub-assembly and rotatable relative to the outer sleeve sub-assembly, and an engagement assembly coaxially extended through the actuation sub-assembly and rotatable relative to each of the actuation sub-assembly and the outer sleeve sub-assembly, the prosthetic valve comprising: a mechanically actuatable frame, a portion of the frame configured to releasably engage with a pair of support arms of a sleeve head of the sleeve sub-assembly and to be braced therebetween; an actuator member comprising a head, the actuation member configured to be rotated in a first direction to radially compress to the frame, and further configured to be rotated in a second direction to radially expand the frame; wherein the head comprises a threaded socket configured for releasable threaded coupling with a threaded member of an engagement head of the engagement sub-assembly, and a faceted outer surface configured for releasable engagement with a faceted socket of an actuation head of the actuation sub-assembly.

In another representative example, a delivery apparatus for implanting a prosthetic valve comprising a mechanically actuatable frame, the frame comprising at least one actuator member configured to be rotated, via rotation of a head thereof, to radially expand and compress the frame, is disclosed. The delivery apparatus comprises at least one actuation assembly configured to be releasably coupled to the frame. The actuation assembly comprises a first member comprising threads disposed on a distal end portion thereof that are configured to form a first releasable connection with a threaded portion of the head of the actuator member, and a second member that is coaxial with the first member and has distal end portion configured to form a second releasable connection with the head of the actuator member. The first member and the second member are rotatable together to rotate the actuator member and expand the prosthetic valve.

In another representative example, a system comprises a prosthetic valve comprising a mechanically actuatable frame, the mechanically actuatable frame comprising at least one actuator member, the actuator member configured to be rotated, via rotation of a head thereof, to radially expand or compress the frame. The system further comprises a delivery apparatus comprising at least one actuation assembly, the actuation assembly comprising a first member comprising a threaded socket at its distal end that is configured to engage with external threads on a threaded portion of the head of the actuator member. The actuation assembly further comprises a second member disposed coaxially within the first member, the second member comprising an actuation head at its distal end that is shaped to mate with and fit within a socket of the head of the actuator member. The first member and the second member are rotatable together to rotate the actuator member and expand the prosthetic valve.

In another representative example, an actuation assembly configured for actuation of a prosthetic valve comprising a mechanically actuatable frame comprising at least one actuator member, the actuator member configured to be rotated, via rotation of a head thereof, to radially expand or compress the frame, is disclosed. The actuation assembly comprises an outer member, where a distal end portion of the outer member comprises a first support extension and a second support extension, an intermediate member coaxially disposed within a lumen of the outer member, where a distal end portion of the intermediate member comprises a first socket configured to interface with a first surface of the head of the actuator member, and an inner member coaxially disposed within a lumen of the intermediate member. A distal end portion of the inner member is configured to interface with a second surface of the head of the actuator member, and the intermediate member and the inner member are rotatable relative to the outer member. The actuation assembly is configured to be transitioned between an engaged position and a disengaged position with the frame.

The above method(s) can be performed on a living animal or on a simulation, such as on a cadaver, cadaver heart, anthropomorphic ghost, simulator (for example, with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

FIG. 1A is a perspective view of a prosthetic device, according to one example.

FIG. 1B is a side elevation view of an exemplary frame of the prosthetic device of FIG. 1A, according to one example.

FIG. 2 is a side elevation view of a delivery apparatus for a prosthetic device, according to one example.

FIGS. 3A-3C are perspective views of a portion of an actuator of the prosthetic device of FIGS. 1A-1B and an actuation assembly of a delivery apparatus, according to one example.

FIGS. 4A and 4B are perspective views of the actuator of FIGS. 3A-3C.

FIGS. 5A and 5B are perspective views of an engagement head of the actuation assembly of FIGS. 3A-3C, according to one example.

FIGS. 6A and 6B are partial cross-sectional views illustrating engagement between the engagement head of the actuator and the actuation assembly of FIGS. 3A-3C.

FIG. 7 is a cross-sectional view illustrating engagement between the engagement head of the actuator and the actuation assembly of FIGS. 3A-3C.

FIGS. 8A and 8B are perspective views of a frame for a prosthetic device and actuator assemblies configured for actuation thereof, according to another example.

FIG. 8C is a detailed perspective view of an exemplary configuration of an apex of the frame of FIGS. 8A and 8B.

FIG. 8D is a detailed perspective view of the apex of the frame coupled with the actuation assembly of FIG. 8A.

FIG. 9 is a perspective view of the exemplary actuation assembly for a delivery apparatus of FIG. 8A.

FIGS. 10A and 10B are front and rear perspective views of an engagement member and threaded member of the actuation assembly of FIG. 8A.

FIG. 11 is a cross-sectional view of the actuator and the actuation assembly of FIG. 8A in an engaged position.

FIGS. 12A and 12B are side elevation views of the actuator and the actuation assembly of FIG. 8A in an engaged position.

FIG. 13 is a perspective view of a portion of an actuator for a prosthetic device, the actuator comprising a head according to another example.

FIG. 14 is an exploded view of an actuation assembly of a delivery apparatus, according to another example, the actuation assembly configured to engage with and actuate the actuator of FIG. 13.

FIGS. 15A-15C are cross-sectional views of the actuator of FIG. 13 and the actuation assembly of FIG. 14 in various stages of engagement.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

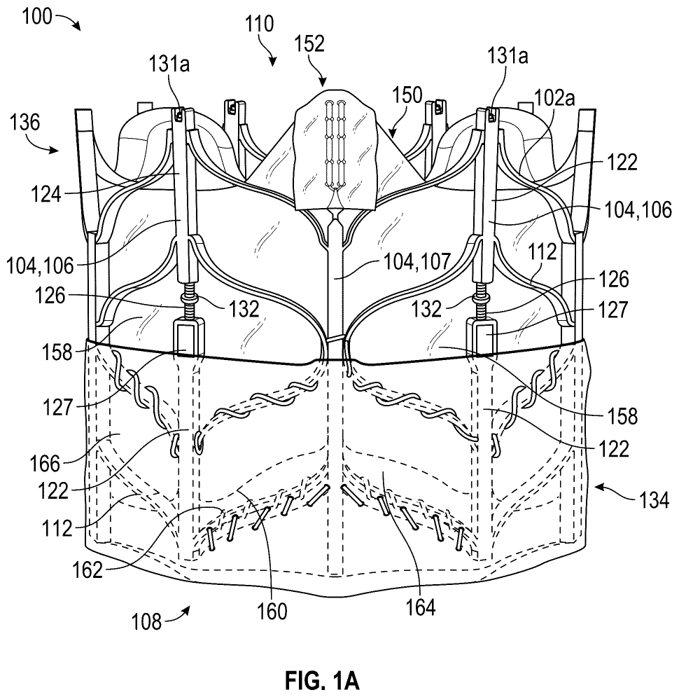

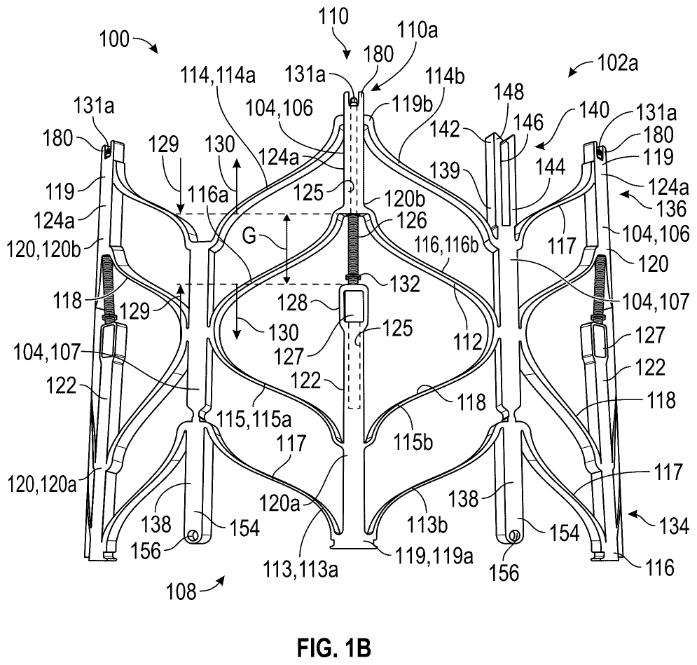

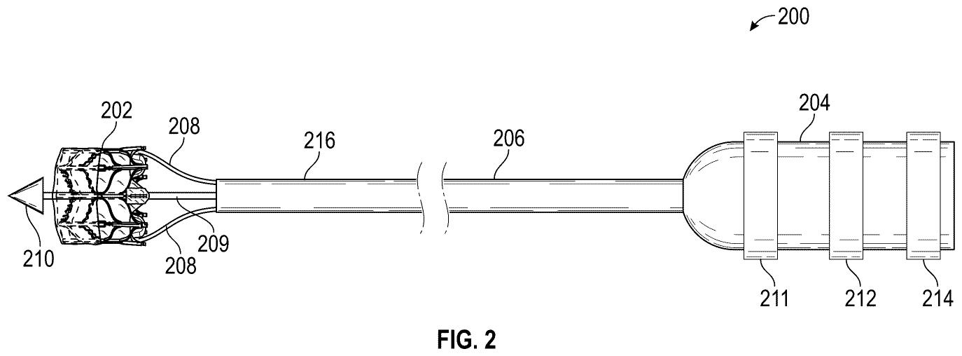

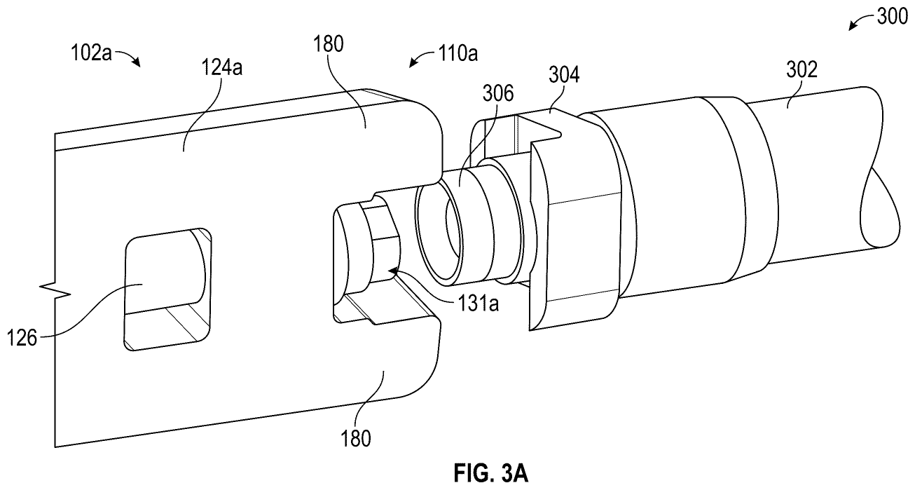

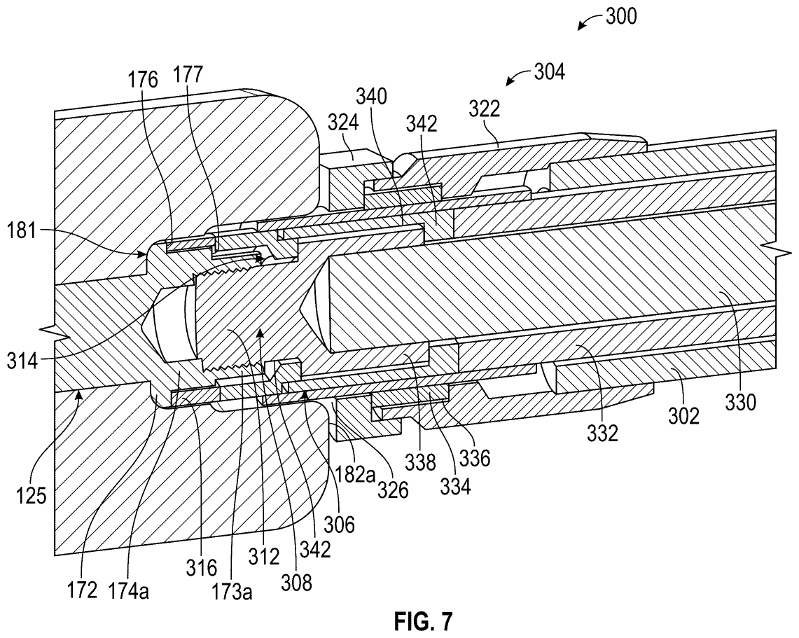

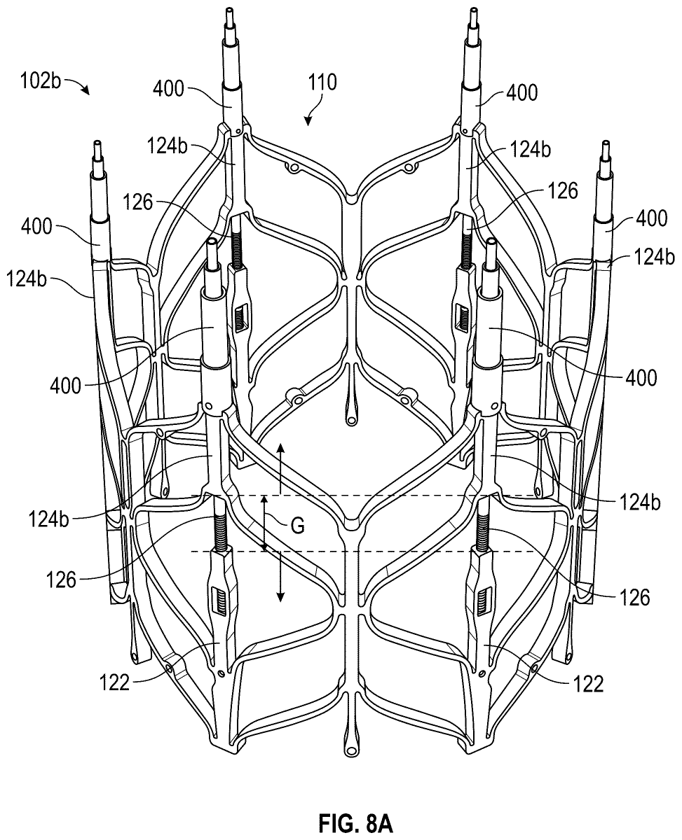

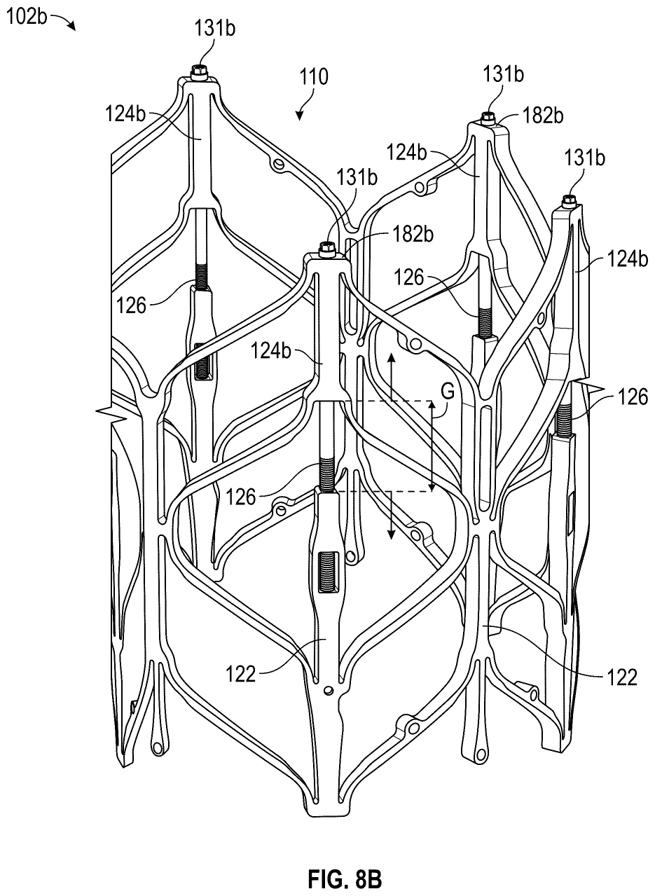

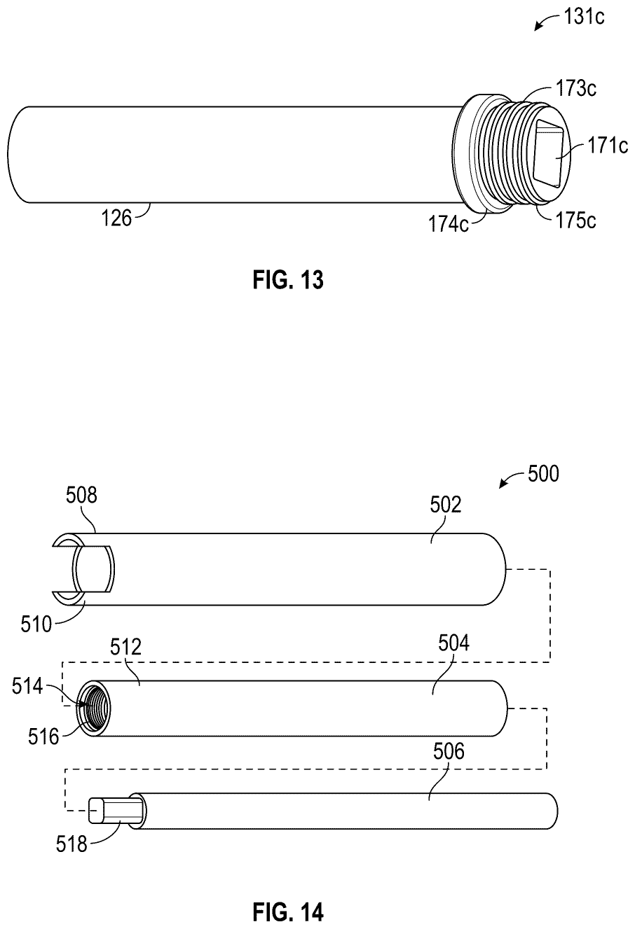

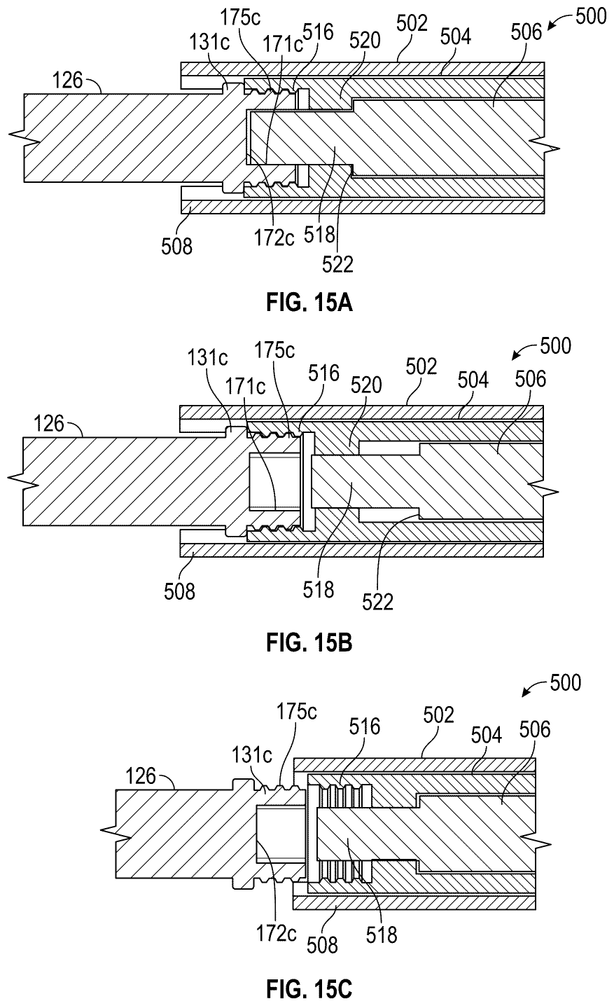

All features described herein are independent of one another and, except where structurally impossible, can be used in combination with any other feature described herein. For example, a prosthetic valve 100 shown in FIG. 1A can be used in combination with a frame 102a (shown in FIGS. 1A and 1B) or a frame 102b (shown in FIGS. 8A and 8B), and the prosthetic valve including one of the foregoing frames can be used in combination with a delivery apparatus 200 (shown in FIG. 2), an actuation assembly 300 (shown in FIGS. 3A-3C and 5A-7), and/or an actuation assembly 400 (shown in FIGS. 8A and 8D-12B), and/or an actuation assembly 500 (shown in FIGS. 14-15C).

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Described herein are examples of delivery apparatuses that can be used to navigate a subject's vasculature to deliver an implantable, expandable medical device (for example, a prosthetic heart valve), tools, agents, or other therapy to a location within the body of a subject. Examples of procedures in which the delivery apparatuses are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In some examples, implant delivery apparatuses disclosed herein can include at least one three-layer delivery and actuation mechanism. For example, a delivery apparatus can include an actuation (or actuator) assembly having three concentric or coaxial sub-assemblies, including an outer sleeve sub-assembly, an intermediate actuation sub-assembly, and an inner engagement sub-assembly. The outer sleeve sub-assembly can include an outer sleeve member and a sleeve head connected at a distal end thereof. The actuation sub-assembly can include an actuation member and an actuation head connected at a distal end thereof. The engagement sub-assembly can include an engagement member and an engagement head connected at a distal end thereof.

In some examples, the actuation member can be coaxial with and disposed within a lumen of the outer sleeve member, and the engagement member can be coaxial with and disposed within a lumen of the actuation member. Each of the actuation member and the engagement member can be rotatable relative to the outer sleeve member, as well as being rotatable relative to each other. Further, the actuation head can be coaxial with and at least partially disposed within a channel of the sleeve head, and the engagement head can be coaxial with and at least partially disposed within a channel of the actuation head. Each of the actuation head and the engagement head can be rotatable relative to the sleeve head, as well as being rotatable relative to each other.

In some examples, the engagement member can be coaxial with and disposed within a lumen of the outer sleeve member, and the actuation member can be coaxial with and disposed within a lumen of the engagement member. Each of the actuation member and the engagement member can be rotatable relative to the outer sleeve member, as well as being rotatable relative to each other. Further, the engagement head can be coaxial with and at least partially disposed within a channel of the sleeve head, and the actuation head can be coaxial with and at least partially disposed within a channel of the engagement head. Each of the actuation head and the engagement head can be rotatable relative to the sleeve head, as well as being rotatable relative to each other.

A proximal end of each of the outer sleeve member, the actuation member, and the engagement member can be coupled to a handle or other controls of the delivery apparatus to enable the surgeon to steer the actuation assembly during transcatheter implant delivery, to control rotation of the engagement member and the engagement head coupled at the distal end thereof, and to control rotation of the actuator member and the actuator head coupled at the distal end thereof.

During use of the actuation assembly, the sleeve head can be utilized as a stationary coupler configured to releasably engage a portion of a frame of a mechanically expandable prosthetic valve. The actuation member and the engagement member can each be utilized as a rotatable coupler configured to releasably engage a respective portion of a head of an actuation member (for example, a threaded member) within the frame. One of the rotatable couplers can be threaded and configured to engage and maintain engagement of the actuation assembly with the frame and the threaded actuation member of the frame, and the other rotatable coupler can be faceted and configured to drive rotation of the threaded actuation member in the frame to cause radial expansion and/or radial compression of the frame.

The delivery apparatus, systems, and actuation assemblies disclosed herein can be configured to limit unwanted axial and rotational movement of the prosthetic heart valve during radial expansion or compression of the frame, and/or during separation of the actuation assembly from the prosthetic valve after radial expansion of the frame. Exemplary delivery apparatus and actuation assemblies and associated methods of use are discussed in further detail elsewhere herein.

Also described herein are prosthetic devices that can be advanced through a patient's vasculature on a delivery apparatus. The prosthetic devices can include one or more expansion and locking mechanisms that can be actuated using the delivery apparatus to radially expand the prosthetic device and lock the prosthetic devices in one or more radially expanded states. As one example, the prosthetic devices can be crimped on or retained by the delivery apparatus in a radially compressed state during delivery, and then radially expanded (and axially shortened) to a radially expanded state once the prosthetic devices reach the implantation site. It is understood that the prosthetic devices disclosed herein may be used with a variety of implant delivery apparatus.

FIGS. 1A-1B illustrate an exemplary prosthetic device (for example, prosthetic heart valve) that can be advanced through a patient's vasculature, such as to a native heart valve, by a delivery apparatus, such as the exemplary delivery apparatus shown in FIG. 2. The frame of the prosthetic device can include mechanical expansion and locking mechanisms that can be integrated into the frame—specifically, into axially extending posts of the frame. The mechanical expansion and/or locking mechanisms can be removably coupled to, and/or actuated by, the delivery apparatus. Specifically, the mechanical expansion and/or locking mechanisms can be removably coupled to an actuation assembly (such as one of the actuation assemblies 300, 400, or 500 respectively illustrated in FIGS. 3A-3C and 5A-7, FIGS. 8A and 8D-12B, and FIGS. 13-15C) of the delivery apparatus, which in turn can be actuated by a physician by adjusting and/or manipulating one or more input devices (for example, one or more knobs, buttons, drawstrings, etc.) that can be included on a handle of the delivery apparatus.

### Examples of the Disclosed Technology

FIGS. 1A and 1B illustrate an example of a prosthetic device 100 (which also may be referred to herein as “prosthetic heart valve 100” and/or “prosthetic valve 100”). Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 100 comprises a frame 102a. FIG. 1A shows the frame 102a with a valvular structure (for example, leaflets) and an optional skirt assembly, while FIG. 1B shows the frame 102a with these components removed. While only one side of the frame 102a is depicted in FIG. 1B, it should be appreciated that the frame 102a forms an annular structure having an opposite side that is substantially identical to the portion shown in FIG. 1B.

The frame 102a comprises a plurality of axially extending posts 104, one or more of which can be configured as integral expansion and locking mechanisms or actuators 106 and/or one or more of which can be configured as support posts 107. Specifically, the actuators 106 (which can be used to radially expand and/or radially compress the prosthetic device 100) can be integrated into the frame 102a of the prosthetic device 100, thereby reducing the crimp profile and/or bulk of the prosthetic device 100 (making the prosthetic device 100 have an overall lower profile). Integrating the expansion and locking mechanisms 106 into the frame 102a can also simplify the design of the prosthetic device 100, making the prosthetic device 100 less expensive and/or easier to manufacture.

The frame 102a has a distal end 108 and a proximal end 110. In some examples, such as the example shown in FIGS. 1A and 1B, the distal end 108 can be an inflow end and the proximal end can be an outflow end, such as when the prosthetic device 100 is configured to replace a native aortic valve and the prosthetic device 100 is delivered to the native aortic valve via a retrograde, transfemoral delivery approach (for example, through a femoral artery and the aorta). However, in other examples, the distal end 108 can be the outflow end and the proximal end 110 can be the inflow end, such as when the prosthetic device 100 is delivered to the native aortic valve via a transapical delivery approach, or when the prosthetic valve is configured to replace a native mitral valve and is delivered to the native mitral valve in a trans-septal delivery approach in which the delivery apparatus and the prosthetic valve are advanced into the right atrium, through the atrial septum, and into the left atrium, wherein the right atrium may be accessed via a femoral vein and inferior vena cava or via the superior vena cava.

The posts 104 can be coupled together by a plurality of circumferentially extending link members or struts 112. Each strut 112 extends circumferentially between adjacent posts 104 to connect all of the axially extending posts 104. As one example, the prosthetic device 100 can include equal numbers of support posts 107 and expansion and locking mechanisms 106 and the expansion and locking mechanisms 106 and the support posts 107 can be arranged in an alternating order such that each strut 112 is positioned between one of the expansion and locking mechanisms 106 and one of the support posts 107 (that is, each struts 112 can be coupled on one end to one of the posts 104 that is configured as one of the expansion and locking mechanisms 106 and can be coupled on the other end to one of the support posts 107). However, the prosthetic device 100 can include different numbers of support posts 107 and expansion and locking mechanisms 106 and/or the expansion and locking mechanisms 106 and the support posts 107 can be arranged in a non-alternating order, in other examples.

As illustrated in FIG. 1B, the struts 112 can include a first row of struts 113 at or near the distal end 108 of the prosthetic device 100, a second row of struts 114 at or near the proximal end 110 of the prosthetic device 100, and third and fourth rows of struts 115, 116, respectively, positioned axially between the first and second rows of struts 113, 114. The struts 112 can form and/or define a plurality of cells (that is, openings) in the frame 102a. For example, the struts 113, 114, 115, and 116 can at least partially form and/or define a plurality of first cells 117 and a plurality of second cells 118 that extend circumferentially around the frame 102a. Specifically, each first cell 117 can be formed by two struts 113a, 113b of the first row of struts 113, two struts 114a, 114b of the second row of struts 114, and two of the posts 104. Each second cell 118 can be formed by two struts 115a, 115b of the third row of struts 115 and two struts 116a, 116b of the fourth row of struts 116. As illustrated in FIG. 1B, each second cell 118 can be disposed within one of the first cells 117 (that is, the struts 115a-116b forming the second cells 118 are disposed between the struts forming the first cells 117 (that is, struts 113a, 113b and the struts 114a, 114b), closer to an axial midline of the frame 102a than the struts 113a-114b).

As illustrated in FIG. 1B, the struts 112 of frame 102a can comprise a curved shape. Each of the first cells 117 can have an axially-extending elliptical shape including first and second apices 119 (for example, proximal apex 119a and distal apex 119b) disposed at the major vertices of the ellipse. In the present example, the distal apex 119 of each of the first cells 117 can include a pair of arms 180 that extend proximally from the proximal end 110 of the frame 102a. For example, the pair of arms 180 can be disposed on a proximal end 110a of a proximal post 124a and extend past a head 131a of an actuator member or a threaded rod 126, such that the head 131a is disposed therebetween and is recessed relative to the proximal end 110 of the frame 102a. Exemplary prosthetic valve frames including proximal posts having a recess (for example, a recess at the proximal end of the post defined by a pair of arms or extensions) that is configured to receive or house a head of a threaded rod and that can be used in combination with the exemplary prosthetic devices and actuation and/or delivery assemblies disclosed herein are described in U.S. Provisional Patent Application No. 63/277,959, which is incorporated by reference herein.

Each second cell 118 can have a circumferentially-extending elliptical shape including first and second apices 120 (for example, proximal apex 120a and distal apex 120b) disposed at the minor vertices of the ellipse. In some examples, the frame 102a comprises six first cells 117 extending circumferentially in a row, six second cells 118 extending circumferentially in a row within the six first cells 117, and thirteen posts 104. However, in some examples, the frame 102a can comprise a greater or fewer number of first cells 117 and a correspondingly greater or fewer number of second cells 118 and posts 104.

Some of the posts 104, such as the posts that are configured as the integral expansion and locking mechanisms 106, can be discontinuous and can each include a first (distal) strut or post 122 and a second (proximal) strut or post 124a that are axially separated from one another by a gap G (FIG. 1B). The first post 122 (that is, the lower post shown in FIG. 1B) can extend axially from the distal end 108 of the prosthetic device 100 toward the second post 124a, and the second post 124a (that is, the upper post shown in FIG. 1B) can extend axially from the proximal end 110 of the frame 102a toward the first post 122. Each first or distal post 122 can be axially aligned with a corresponding second or proximal post 124a and one or both of the posts 122, 124a can include an inner bore 125, so that, for example, the axially aligned pair of posts 122, 124a can receive an actuator member, such as in the form of a substantially straight threaded rod 126 as shown in the illustrated example. The threaded rod 126 also may be referred to herein as an actuator, an actuator member, and/or a screw actuator.

The threaded rod 126 can be coupled to the distal post 122 and/or the proximal post 124a. In some examples, the rod 126 can be rotatably coupled to the distal post 122 such that the rod 126 can only be rotated relative to the distal post 122 to move the rod 126 axially relative to the distal post but otherwise cannot move relative to the distal post 122 (for example, the threaded rod 126 cannot slide axially relative to the distal post 122 or move circumferentially and/or radially relative to the distal post 122). Further, the rod 126 can be restrained radially and/or circumferentially by the proximal post 124a such that the rod 126 can only be rotated and/or slid axially relative to the proximal post 124a but otherwise cannot move relative to the proximal post 124a (for example, radially and/or circumferentially). As one example, the threaded rod 126 can be inserted through the inner bore 125 of one of the proximal posts 124a and into a stationary nut 127 and/or bore 125 included in an axially aligned one of the distal posts 122. However, in other examples, the threaded rod 126 need not extend through a bore in the proximal post 124a and instead can be restrained and/or restrained by the proximal post 124a using other suitable structures such as guides, straps, loops, collars, etc.

The stationary nut 127 can be included at a proximal end portion 128 of the distal post 122. In some examples, the threaded rod 126 can extend distally past the nut 127 into the inner bore 125 of the distal post 122. The stationary nut 127 can be held in a fixed position relative to the distal post 122 such that the nut 127 does not rotate relative to the distal post 122. In this way, whenever the threaded rod 126 is rotated (for example, by a physician) the threaded rod 126 can rotate relative to both the stationary nut 127 and the distal post 122. The nut 127 can include a threaded bore that that is configured to engage the threads of the threaded rod 126 to prevent the rod 126 from moving axially relative to the nut 127 and the post 122 unless the threaded rod 126 is rotated relative to the nut 127. Thus, the threaded rod 126 can be retained/held by the nut 127 and can only be moved relative to the nut 127 and/or the post 122 by rotating the threaded rod 126 relative to the nut 127 and/or the post 122. In some examples, in lieu of using the nut 127, at least a portion of the inner bore 125 of the distal post 122 can be threaded. For example, the proximal end portion 128 of the distal post 122 can comprise inner threads configured to engage the threaded rod 126 such that rotation of the threaded rod causes the rod 126 to move axially relative to the first post 122.

When the threaded rod 126 is inserted into and/or otherwise coupled to a pair of axially aligned posts 122, 124a, the pair of axially aligned posts can serve as one of the expansion and locking mechanisms 106. In some examples, one of the threaded rods 126 can be inserted through each pair of axially aligned posts 122, 124a so that all of the posts 122, 124a serve as expansion and locking mechanisms 106. As just one example, the prosthetic device 100 can include six pairs of posts 122, 124a, and each of the six pairs of posts 122, 124a can be configured as one of the expansion and locking mechanisms 106 for a total of six expansion and locking mechanisms 106. In some examples, not all pairs of posts 122, 124a need be expansion and locking mechanisms (that is, actuators). If a pair of posts 122, 124a is not used as an actuator, a threaded rod 126 need not extend through the posts 122, 124a of that pair. In the description that follows, the first and second (or distal and proximal) posts 122, 124a, respectively, that are used as actuators (that is, those that include threaded rods 126), can be referred to as first and second (or distal and proximal) actuator posts 122, 124a, or more simply, first and second (or distal and proximal) posts 122, 124a.

The threaded rod 126 can be rotated relative to the nut 127 and/or the first post 122 to axially foreshorten and/or axially elongate the frame 102a, thereby radially expanding and/or radially compressing, respectively, the prosthetic device 100. Specifically, when the threaded rod 126 is rotated relative to the nut 127 and/or the first post 122, the first and second posts 122, 124a can move axially relative to one another, thereby widening or narrowing the gap G (FIG. 1B) separating the posts 122, 124a, and thereby radially compressing or radially expanding the prosthetic device 100, respectively. Thus, the gap G (FIG. 1B) between the first and second posts 122, 124a narrows as the frame 102a is radially expanded and widens as the frame 102a is radially compressed. In other words, rotation of the threaded rod 126 in a first direction (for example, clockwise) can cause corresponding axial movement of the first and second posts 122, 124a toward one another (as shown by arrows 129 in FIG. 1B), thereby radially expanding the frame 102a, while rotation of the threaded rod 126 in an opposite second direction (for example, counterclockwise) causes corresponding axial movement of the first and second posts 122, 124a away from one another (as shown by arrows 130 in FIG. 1B), thereby radially compressing the frame.

The threaded rod 126 can extend proximally past the proximal end of the proximal post 124a and can include the head 131a at its proximal end that can serve at least two functions. First, as will be described in greater detail below with reference to FIGS. 3A-7 (as well as in the related discussions of FIGS. 8A-12B and 13-15C), the head 131a can releasably couple the threaded rod 126 to a respective actuation assembly of the delivery apparatus that can be used to drive rotation of the head 131a and the threaded rod 126 to radially expand and/or radially compress the prosthetic device 100. Second, the head 131a can prevent the proximal post 124a from moving proximally relative to the threaded rod 126 and can apply a distally directed force to the proximal post 124a, such as when radially expanding the prosthetic device 100. Specifically, the head 131a can have a width greater than a diameter of the inner bore 125 of the proximal post 124a such that the head 131a is prevented from moving into the inner bore 125 of the proximal post 124a. Thus, as the threaded rod 126 is threaded farther into the nut 127, the head 131a of the threaded rod 126 draws closer to the nut 127 and the first post 122, thereby drawing the proximal post 124a towards the distal post 122, and thereby axially foreshortening and radially expanding the prosthetic device 100.

The threaded rod 126 also can include a stopper 132 (for example, in the form of a nut or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed position on the threaded rod 126 such that it moves in lockstep with the threaded rod 126. The stopper 132 can apply a proximally directed force to the proximal post 124a to radially compress the prosthetic device 100. Specifically, during crimping/radial compression of the prosthetic valve 100, the threaded rod 126 can be rotated in the second direction (for example, counterclockwise) causing the stopper 132 to push against (that is, provide a proximally directed force to) the distal end of the proximal post 124a, thereby causing the proximal post 124a to move away from the distal post 122, and thereby axially elongating and radially compressing the prosthetic device 100.

Thus, the proximal posts 124a can be axially retained and/or restrained between the head 131a of the threaded rod 126 and the stopper 132. That is, each proximal post 124a can be (alternatively) restrained at its proximal end by the head 131a of the threaded rod 126 and at its distal end by the stopper 132. In this way, the head 131a can apply a distally directed force to the proximal post 124a to radially expand the prosthetic device 100 when the head 131a is rotated in the first direction (for example, clockwise), while the stopper 132 can apply a proximally directed force to the proximal post 124a to radially compress the prosthetic device 100 when the head 131a is rotated in the second direction (for example, counterclockwise). As explained above, radially expanding the prosthetic device 100 axially foreshortens the prosthetic device 100, causing a distal end portion 134 and a proximal end portion 136 of the prosthetic device 100 to move towards one another axially, while radially compressing the prosthetic device 100 axially elongates the prosthetic device 100, causing the distal and proximal end portions 134, 136 to move away from one another axially.

As also introduced above, some of the posts 104 can be configured as support posts 107 (also referred to as “axial struts”). As shown in FIG. 1B, the support posts 107 can extend axially between the distal and proximal end portions 134, 136 of the frame 102a and can have a distal end portion 138 and a proximal end portion 139. The proximal end portion 139 of one or more support posts 107 can include a commissure support structure or member 140. The commissure support member 140 can comprise first and second commissure arms 142, 144 defining a commissure opening 146 between them. The proximal end of each commissure arm 142, 144 can include a tooth 148 extending into the commissure opening 146. The commissure opening 146 can extend radially through a thickness of the post 107 and can be configured to accept a portion of a valvular structure 150 (for example, a commissure 152) to couple the valvular structure 150 to the frame 102a. For example, each commissure 152 can be mounted to a respective commissure support member 140, such as by inserting a pair of commissure tabs of adjacent leaflets through the opening 146 and suturing the commissure tabs to each other and/or the arms 142, 144. In some examples, the opening 146 can be fully enclosed by the post 107 (for example, not extending to the proximal edge) such that a portion of the valvular structure 150 can be slid radially (rather than axially) into the commissure opening 146. The teeth 148 can help retain the commissure 152 within the commissure opening 146. In the illustrated example, the commissure opening 146 has a substantially rectangular shape and extends to the distal end of the post 104. However, in some examples, the commissure opening can have any of various shapes (for example, square, oval, square-oval, triangular, L-shaped, T-shaped, C-shaped, etc.).

Though only one support post 107 comprising a commissure support member 140 is shown in FIG. 1B, it should be noted that the frame 102a can comprise any number of support posts 107, any number of which can be configured as commissure support members 140. For example, the frame 102a can comprise six support posts 107, three of which are configured as commissure support members 140. However, in some examples, the frame 102a can comprise more or less than six support posts 107 and/or more or less than three commissure support members 140.

The opposite distal end portion 138 of each support post 107 can comprise an extension 154 that extends toward the distal end of the frame 102a. The extension 154 can comprise an aperture 156 extending radially through a thickness of the extension 154. In some examples, the extension 154 can extend such that a distal edge of the extension 154 aligns with or substantially aligns with the distal end of the frame 102a. In use, the extension 154 can prevent or mitigate portions of an outer skirt from extending radially inwardly and thereby prevent or mitigate any obstruction of flow through the distal end portion 134 of the frame 102a caused by the outer skirt. The extensions 154 can further serve as supports to which portions of the inner and/or outer skirts can be coupled. For example, sutures used to connect the inner and/or outer skirts can be wrapped around the extensions 154 and/or can extend through openings 156.

The frame 102a can be a unitary and/or fastener-free frame that can be constructed from a single piece of material (for example, Nitinol). The plurality of cells can be formed by removing portions (for example, via laser cutting) of the single piece of material. The threaded rods 126 can then be inserted through the bores in the proximal posts 124a and threaded into the threaded nut 127.

As illustrated in FIG. 1A, the prosthetic valve 100 can further include the valvular structure 150, which is coupled to and supported inside the frame 102a. The valvular structure 150 is configured to regulate the flow of blood through the prosthetic valve 100, from the inflow end to the outflow end. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form the commissures 152, each of which can be secured to a respective post 104 (for example, to a support post 107) and/or to other portions of the frame 102a.

In the example depicted in FIG. 1A, the valvular structure 150 includes three leaflets 158, which can be arranged to collapse in a tricuspid arrangement. Each leaflet 158 can have a proximal edge portion 160. As shown in FIG. 1A, the proximal edge portions 160 of the leaflets 158 can define an undulating, curved scallop edge that follows or tracks portions of the struts 112 of frame 102a in a circumferential direction when the frame 102a is in the radially expanded configuration. The proximal edges 160 of the leaflets can be referred to as a “scallop line.”

As shown in FIG. 1A, the proximal edge portions 160 of the leaflets 158 can be sutured to an inner skirt 164 generally along the scallop line. The inner skirt 164 can in turn be sutured, via one or more sutures 162, for example, to adjacent struts 112 of the frame 102a. In some examples, the leaflets 158 can be sutured directly to the frame 102a along the scallop line.

The prosthetic valve 100 can further include one or more skirts or sealing members. For example, the prosthetic valve 100 can include the inner skirt 164, mounted on the radially inner surface of the frame 102a. The inner skirt 164 can function as a sealing member to prevent or decrease perivalvular leakage, to anchor the leaflets 158 to the frame 102a, and/or to protect the leaflets 158 against damage caused by contact with the frame 102a during crimping and during working cycles of the prosthetic device 100. The prosthetic device 100 can further include an outer skirt 166 mounted on the outer surface of the frame 102a. The outer skirt 166 can function as a sealing member for the prosthetic device 100 by sealing against the tissue of the native valve annulus and helping to reduce paravalvular leakage past the prosthetic device 100. The inner and outer skirts 164, 166 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valves can be found, for example, in U.S. Patent Application No. 62/854,702 and PCT Patent Application No. US2020/024559, each of which is incorporated by reference herein.

As introduced above, the head 131a of each of the threaded rods 126 can be releasably coupled to a corresponding actuation assembly of a delivery apparatus. In some examples, one or more other portions of the frame 102a can also releasably couple the prosthetic device 100 to an actuation assembly of a delivery apparatus. Referring to FIG. 2, an exemplary delivery apparatus 200 for delivering a prosthetic device or valve 202 (which may have the configuration of for example, the prosthetic device 100 or another configuration) to a desired implantation location is illustrated therein. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts. It will be appreciated the prosthetic valve 202 can have the configuration of the prosthetic device 100 shown in FIGS. 1 and 2 or another configuration. For example, the prosthetic valve 202 can have a configuration similar those described in U.S. Provisional Patent Application Nos. 63/085,947 and/or 63/277,959, each of which is incorporated by reference herein.

In the illustrated example, the delivery apparatus 200 generally includes a handle 204, a first elongated shaft 206 (which comprises an outer shaft in the illustrated example) extending distally from the handle 204, at least one actuation assembly 208 extending distally through the outer shaft 206, a second elongated shaft 209 (which comprises an inner shaft in the illustrated example) extending through the first shaft 206, and a nosecone 210 coupled to a distal end portion of the second shaft 209. The inner shaft 209 and the nosecone 210 can define a guidewire lumen for advancing the delivery apparatus through a patient's vasculature over a guidewire. The at least one actuation assembly 208 can be configured to radially expand and/or radially collapse the prosthetic valve 202 when actuated, such as by user operation of one or more knobs 210, 212, 214 included on the handle 204 of the delivery apparatus 200.

Though the illustrated example shows two actuator assemblies 208 for purposes of illustration, it should be understood that one actuator 208 can be provided for each actuator (for example, threaded rod 126) on the prosthetic valve. For example, three actuator assemblies 208 can be provided for a prosthetic valve having three actuators. In other examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve 202 during delivery thereof to the implantation site.

The actuator assemblies 208 can be releasably coupled to the prosthetic valve 202. In the illustrated example, each actuation assembly 208 can be coupled to a respective actuator of the prosthetic valve 202 (such as being coupled to for example, the head 131a of the threaded rod 126 and/or other portions of the frame 102a). Each actuation assembly 208 can comprise at least a support tube and an actuator member. When actuated, the actuation assembly can transmit torque, pushing, and/or pulling forces (or other forces and/or combinations thereof) to portions of the prosthetic valve to radially expand and collapse the prosthetic valve as previously described. The actuator assemblies 208 can be at least partially disposed radially within, and extend axially through, one or more lumens of the outer shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand, position, adjust, and/or deploy the prosthetic valve 202. For example, the handle 204 comprises first, second, and third knobs 210, 212, and 214 that can be utilized to control the delivery apparatus 200.

In an illustrative example, the first knob 211 can be a rotatable knob configured to produce axial movement of the outer shaft 206 relative to the prosthetic valve 202 in the distal and/or proximal directions in order to deploy or deliver the prosthetic valve 202 from the delivery sheath 216 once the prosthetic valve 202 has been advanced to a location at or adjacent the desired implantation location with the patient's body. For example, rotation of the first knob 211 in a first direction (for example, clockwise) can result in retraction of the sheath 216 proximally relative to the prosthetic valve 202 and rotation of the first knob 211 in a second direction (for example, counterclockwise) can cause advancement of the sheath 216 distally. In other examples, the first knob 211 can be actuated by sliding or moving the knob 211 axially, such as pulling and/or pushing the knob. In other examples, actuation of the first knob 211 (rotation or sliding movement of the knob 211) can cause, produce, and/or result in axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can cause, produce, and/or result in rotation of the threaded rods of the prosthetic valve 202 via the actuator assemblies 208, as will be described in greater detail below with reference to FIGS. 3A-7, as well as FIGS. 8A-12B. In some examples, rotation of the second knob 212 in a first direction (for example, clockwise) can radially expand the prosthetic valve 202 and rotation of the second knob 212 in a second direction (for example, counterclockwise) can radially collapse the prosthetic valve 202. In other examples, the second knob 212 can be actuated by sliding or moving the knob 212 axially, such as pulling and/or pushing the knob.

The third knob 214 can be a rotatable knob operatively connected to a proximal end portion of each actuation assembly 208. The third knob 214 can be configured to retract an outer sleeve or support tube of each actuation assembly 208 to disconnect the actuator assemblies 208 from the proximal portions of the actuators of the prosthetic valve (for example, threaded rod), as further described below. Once the actuator assemblies 208 are uncoupled from the prosthetic valve 202, the delivery apparatus 200 can be removed from the patient, leaving just the prosthetic valve 202 in the patient. Additional exemplary features for a delivery apparatus and methods of using a delivery apparatus that can be used in combination with the delivery apparatus 200 and the prosthetic valves disclosed herein are described in U.S. Pat. No. 9,827,093, which is incorporated by reference herein.

Turning now to FIGS. 3A-7, an exemplary actuation assembly 300 of a delivery apparatus configured to be releasably coupled to the frame 102a for actuation thereof is shown and described. The actuation assembly 300 can be, for example, one of the actuator assemblies 208 of the delivery apparatus 200 shown in FIG. 2. The frame 102a can be utilized in the prosthetic valve 100 shown in FIG. 1A or other prosthetic valves.

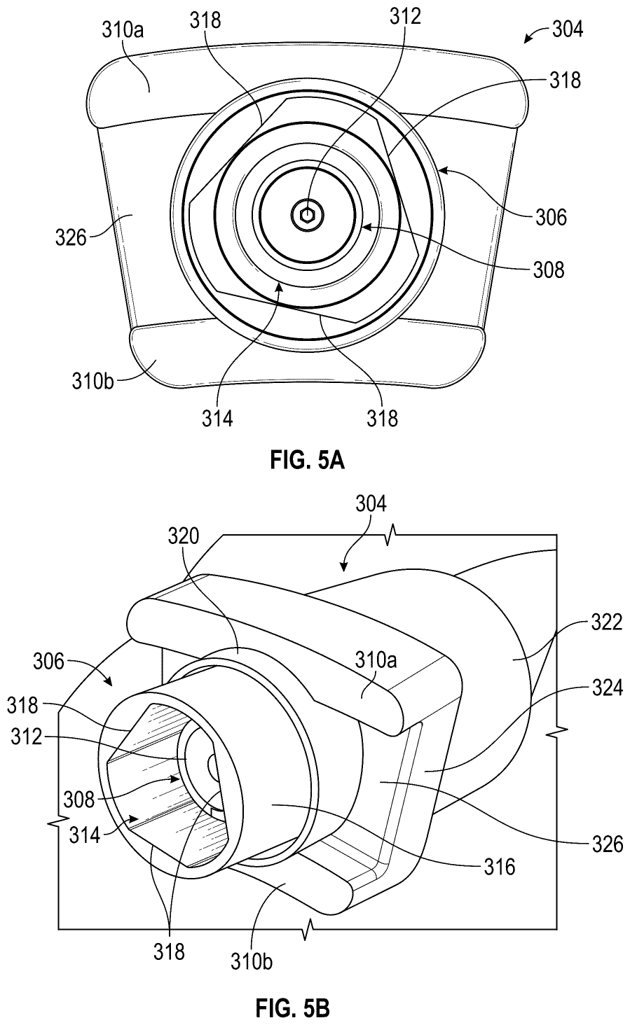

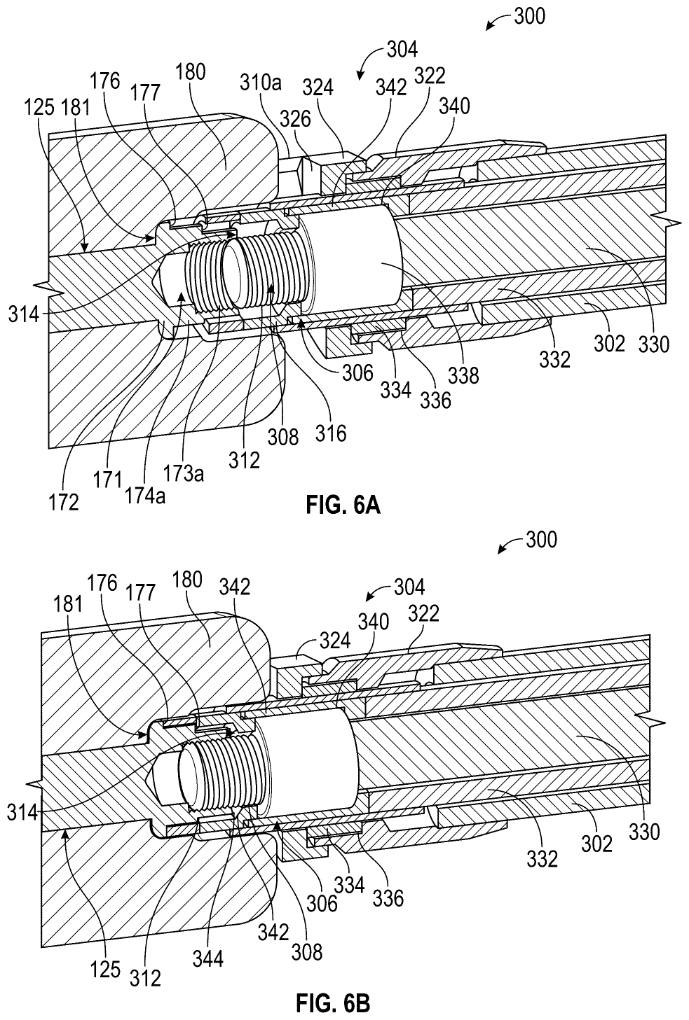

As best illustrated in FIGS. 6A-7, the actuation assembly 300 includes three concentric or coaxial sub-assemblies, including an outer sleeve sub-assembly 301, an intermediate actuator sub-assembly 303, and an inner engagement sub-assembly 305. The outer sleeve sub-assembly 301 can include an outer sleeve member or support member 302 and a sleeve head 304 connected at a distal end thereof. The actuator sub-assembly 303 can include an actuator member 332 (or actuation member) and an actuator head 306 connected at a distal end thereof. The engagement sub-assembly 305 can include an engagement member 330 and an engagement head 308 connected at a distal end thereof.

The actuation member 332 can be coaxial with and disposed within a lumen of the outer sleeve member 302, and the engagement member 330 can be coaxial with and disposed within a lumen of the actuation member 332. Each of the actuation member 332 and the engagement member 330 can be rotatable relative to the outer sleeve member 302. Further, each of the actuation member 332 and the engagement member 330 can be rotatable relative to each other.

The sleeve member 302, the actuation member 332, and the engagement member 330 can each comprises an elongated flexible shaft having any of various constructions and can be made from any of various materials. For example, the sleeve member 302, the actuation member 332, and/or the engagement member 330 can comprise a flexible shaft comprising one of more polymeric layers and optionally one or more reinforcing layers embedded within or surrounding one or more the polymeric layers. The reinforcing layers can comprise a braided layer and/or a layer comprising a helical coil. In some examples, the engagement member 330 can comprise a cable, wire, or flexible rod.

Also illustrated in FIGS. 6A-7, the actuation head 306 can be coaxial with and at least partially disposed within a channel of the sleeve head 304, and the engagement head 308 can be coaxial with and at least partially disposed within a channel of the actuation head 306. Each of the actuation head 306 and the engagement head 308 can be rotatable relative to the sleeve head 304. Further, each of the actuation head 306 and the engagement head 308 can be rotatable relative to each other.

Although not specifically illustrated, a proximal (opposing) end portion of each of the outer sleeve member, the actuation member, and the engagement member can be operatively coupled to a handle or other controls of the delivery apparatus to enable the physician to control rotation of the engagement member and the engagement head coupled at the distal end thereof and/or to control rotation of the actuator member and the actuator head coupled at the distal end thereof. For example, the proximal end portion of the actuator member 332 of each assembly 300 can be operatively coupled to the knob 212 (FIG. 2), which is configured to rotate all of the actuator members 332 together upon rotation of the knob 212 to control radial expansion or compression of the prosthetic valve. The proximal end portion of the engagement member 330 of each assembly 300 can be operatively coupled to the knob 214 (FIG. 2), which is configured to rotate all of the engagement members 330 together to connect or disconnect the engagement heads 308 from the threaded rods 126 upon rotation of the knob 214.

FIGS. 3A and 3B illustrate the actuation assembly 300 detached from one of the threaded rods 126 and one of the proximal posts 124a, while FIG. 3C illustrates the actuation assembly 300 releasably coupled to or engaged with the threaded rod 126 and the proximal post 124a. As introduced above, the head 131a of each threaded rod 126 in a mechanically expandable frame of a prosthetic device (such as, for example, the prosthetic device 100 and/or the prosthetic device 202) can be configured to couple with an actuation assembly for radial expansion and radial compression of the frame. In some examples, the head 131a comprises a proximal end portion of the threaded rod 126, which extends proximally through an inner bore 125 of the proximal post 124a. As can be seen in FIGS. 3A-3C, at the proximal end 110a thereof, the proximal post 124a (at an apex of the frame) includes two arms or extensions 180 that extend past the proximal end of the head 131a on opposing sides thereof. It will be appreciated that structures and functions of the proximal post 124a, the threaded rod 126, and the head 131a with respect to other portions of the frame, such as the configurations that enable radial expansion and/or compression of the prosthetic valve, are described above with reference to FIGS. 1A and 1B.

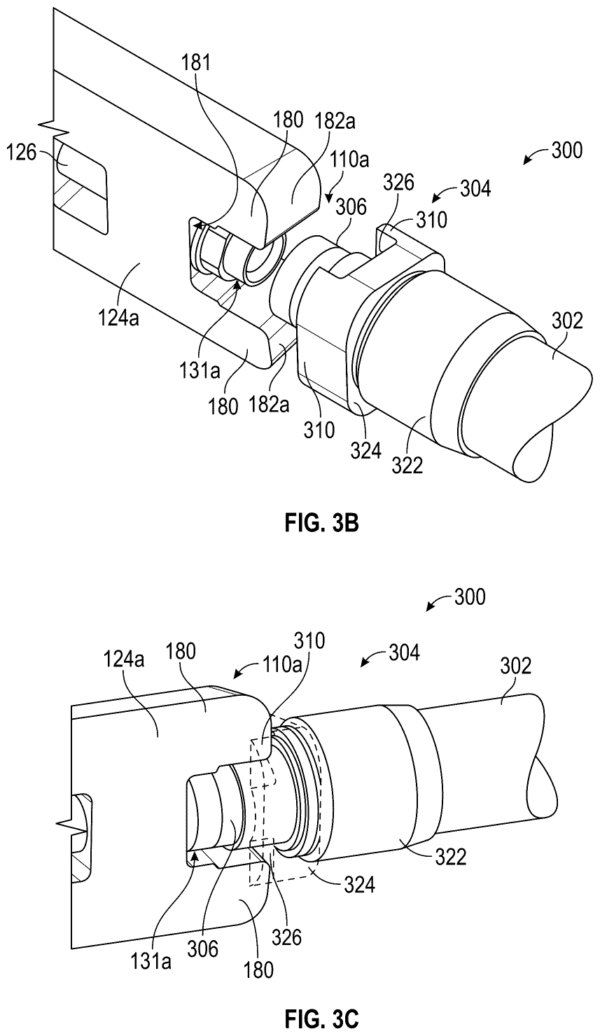

As can be seen in FIGS. 4A and 4B, in the present example, the head 131a can comprise a threaded socket 171 formed by a unitary structure including a base 172 having a first raised wall 173a and a second raised wall 174a, which each extend axially from the base 172. The second raised wall 174a can be characterized as a collar disposed around (and concentric with) a distal portion of the first raised wall 173. The first raised wall can be cylindrical in shape and have a substantially smooth, continuously curved exterior surface, while the second raised wall 174a can include one or more facets or flat portions 175a evenly and circumferentially spaced apart at the exterior surface thereof (such as, for example, including three facets 175a, each positioned at 120° on the exterior surface of the second raised wall 174a relative to others of the facets). The base 172 is a relatively flat circular portion, having a greater diameter than each of the first raised wall 173a and the second raised wall 174a, while the first raised wall 173a has a greater length and a narrower diameter relative to the second raised wall 174a, giving the head 131a an overall stepped configuration. In other words, a first annular shoulder 176 is formed between the base 172 and the first raised wall 173a, and a second annular shoulder 177 is formed between the first raised wall 173a and the second raised wall 174a (FIGS. 6A-7).

As discussed above with reference to FIG. 1B, the head 131a can have an overall width greater than a diameter of the inner bore 125 of the proximal post 124a such that the head 131a is configured to prevent advancement of the head into the inner bore 125 of the proximal post 124a and such that the head 131a abuts a proximal face 181 of the proximal post 124a of the frame. The head 131a can be used to apply a distally-directed force to the proximal post 124a, for example, during radial expansion of a frame (such as, for example, the frame 102a shown in FIGS. 1A and 1B).

Returning to FIGS. 3A-3C, as illustrated therein and described above, each actuation assembly 300 can include the support tube or outer sleeve member 302, the sleeve head 304, the actuator head 306, and the engagement head 308. In some examples, the sleeve head 304 is configured to engage an end portion of the frame 102a (for example, an end portion of the frame adjacent to the sleeve head) through which the threaded rod 126 extends. For example, the sleeve head 304 can be configured to engage a proximal end 110a of the proximal post 124a. The actuator head 306 and the engagement head 308 can be configured to engage the head 131a of the threaded rod 126.

Specifically, the sleeve head 304 can include a pair of support extensions 310 (or prongs) distally extending at a distal end thereof that are configured to releasably engage or releasably mate with opposing surfaces of the end portion of the frame. In the illustrated example, the support extensions 310 are configured to releasably engage opposing surface of the proximal end 110a of the proximal post 124a (for example, at interior and exterior surfaces of the pair of arms 180 and at a proximal face 182a). In some examples, the sleeve head 304 having the pair of support extensions 310 can be characterized as a stationary coupler configured to couple with a portion of the frame.

The actuator head 306 and the engagement head 308 can be configured to releasably engage or releasably mate with respective portions of the head 131a of the threaded rod 126. In some examples, each of the actuator head 306 and the engagement head 308 can be characterized as a rotatable coupler configured to couple to a respective portion of the head of the threaded rod. As the head 131a is recessed between the pair of arms 180, the actuation assembly 300 can have a complementary configuration in which the actuator head 306 extends distally relative to the sleeve head 304, which enables the actuator head 306 and the engagement head 308 to be inserted between the pair of arms 180 for engagement with the head 131a.

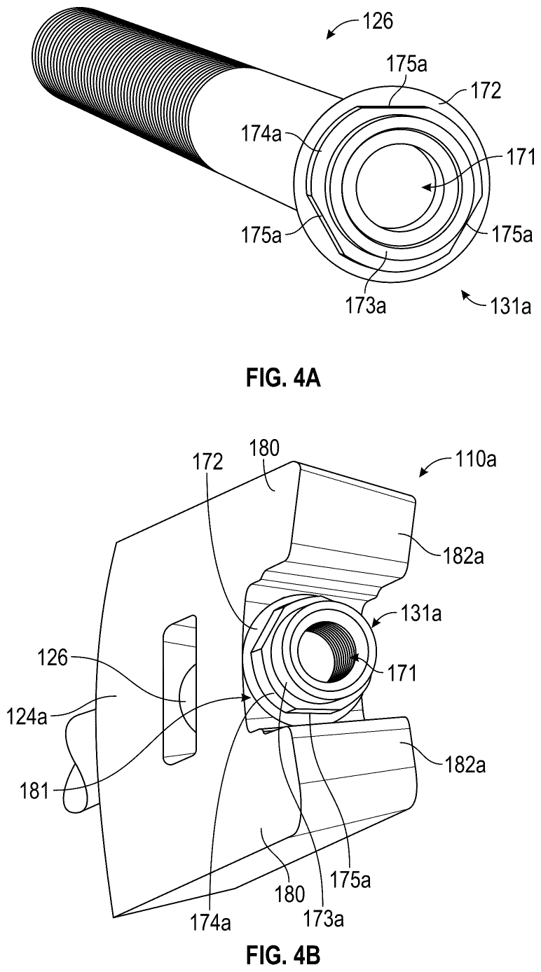

As illustrated in FIGS. 5A and 5B, the actuator head 306 can include a socket 314 formed by or defined by a collar 316, which can have a substantially smooth, continuously curved exterior surface. The engagement head 308, including a central threaded engagement member 312, can be disposed within the socket 314 and can rotate relative to the socket 314. An interior surface of the socket 314 includes one or more facets or flat portions 318 evenly spaced at the interior surface thereof (such as, for example, including three facets, each positioned at 120° on the interior surface relative to others of the facets). It will be appreciated that the configuration of the facets 318 can be complementary to the configuration of the facets 175a to enable engagement therebetween (discussed in further detail below with reference to FIGS. 6A-7). In some examples, the actuator head 306 can further include a cylindrical wall 320 disposed around a proximal end of the collar 316. Each of the collar 316 and the cylindrical wall 320 can have a substantially smooth, continuously curved exterior surface.

Also shown in FIGS. 5A and 5B, the actuator head 306 can be centrally disposed within a channel of the sleeve head 304, between the pair of support extensions 310, and can be rotatable within the channel of the sleeve head 304. The sleeve head 304 can include a proximal portion 322 and a distal portion 324, the distal portion 324 being a base portion having the pair of support extensions 310 extending distally therefrom. In the present example, the proximal portion 322 has a generally cylindrical shape, however, in other examples the proximal portion can have a different shape, such as having a cuboid, a hexagonal prism shape or other shape. The proximal portion 322 can be fixedly attached to a distal end the outer sleeve 302, and the distal portion 324 can be attached to or formed as a unitary structure with the proximal portion 322. In some examples, a width of the distal portion 324 can be greater than a diameter of proximal portion 322. In other examples, a width of the distal portion 324 can be less than or equal to a diameter of proximal portion 322.

As described above, the pair of support extensions 310 can be configured to enable engagement of the sleeve head 304 with a portion of the frame 102a of a prosthetic valve, such as, for example, enabling engagement with the proximal end 110a of the proximal post 124a. In some examples, the pair of support extensions 310 can have a configuration or shape that is complementary to a configuration for shape the proximal end 110a of the proximal post 124a. For example, as illustrated in FIGS. 5A and 5B, the support extensions 310 can be configured to engage with a proximal post having a curvature (which contributes to an overall continuous curvature or cylindrical shape of the prosthetic valve). Accordingly, in the present example, the support extensions include an exterior support extension 310a and an interior support extension 310b, which can have curvatures and lengths configured to respectively match those of the exterior and interior surfaces of the proximal post. For example, the exterior support extension 310a can have greater length than the interior support extension 310b. In other examples, such as the example shown in FIGS. 3A-3C, the surfaces of the proximal post may be generally flat, and the support extensions can have a similar configuration, such as being generally flat (on at least the interior surface thereof) and having equal or similar length.

In either example, the pair of support extensions 310 can be configured to be inserted or slid over the proximal end 110a of the proximal post 124a (for example, slid over the pair of arms 180) so that the interior surfaces of the support extensions 310 abut or contact the exterior and interior surfaces of the proximal end 110a of the proximal post 124a. In some examples, the proximal end 110a of the proximal post 124a is configured to be braced between the pair of support extensions 310 and limit or restrain rotational and/or lateral movement of the sleeve head 304 relative to the proximal post 124a. Further, a proximal face 182a of the proximal post 124a at the pair of arms 180 is configured to abut an interior surface 326 (a surface disposed between the support extensions 310) on the distal portion 324 of the sleeve head 304, thereby limiting axial movement in a proximal to distal direction relative to the proximal post 124a.

As discussed above, FIGS. 3A and 3B show the sleeve head 304 in a disengaged position relative to the proximal post 124a, while FIG. 3C shows the sleeve head 304 engaged with the proximal post 124a. More specifically, in FIG. 3A, the sleeve head 304 is aligned for engagement with the proximal post 124a, and in FIG. 3C, the sleeve head 304 is in a position where the proximal end of the pair of arms 180 is inserted and/or braced between the support arms 310 and contacts the interior surface 326 of the distal portion 324 of the sleeve head 304. For illustrative purposes, the distal portion 324 of the sleeve head 304 is shown as transparent in FIG. 3C.

The partial cross-sectional views of FIGS. 6A and 6B and the cross-sectional view of FIG. 7 illustrate engagement or releasable coupling of the sleeve head 304 with the distal end 110 of the proximal post 124a and the actuator head 306 with the head 131a of the threaded rod 126. Specifically, FIG. 6A depicts a state where the sleeve head 304 and the actuator head 306 are partially engaged. As discussed above and shown in FIG. 6A, the proximal end 110 of the proximal post 124a can be braced between the pair of support extensions 310 in order limit or restrain rotational and/or lateral movement of the sleeve head 304 relative to the proximal post 124a. Additionally, the first raised wall 173a of the head 131a can be fitted within the socket 314 of the actuator head 306, such that the threaded engagement member 312 is aligned with the threaded socket 171 of the head 131a.

FIGS. 6B and 7 depict a state where the sleeve head 304, the actuator head 306, and the engagement head 308 are fully engaged. As discussed above and shown in FIGS. 6B and 7, the proximal face 182a of the proximal post 124a at the pair of arms 180 can abut the interior surface 326 (the surface disposed between the support extensions 310), thereby limiting axial movement in the proximal to distal direction of the sleeve head 304 relative to the proximal post 124a. Further, in the fully engaged state, the facets 318 within the socket 314 of the actuator head 306 can engage the facets 175a on the exterior surface of the second raised wall 174a of the head 131a, and the threaded engagement member 312 can be threaded into the threaded socket 171 of the head 131a.

As can be seen in FIGS. 6A-7, the actuator head 306 can include a circumferential or annular protrusion 334 (on an exterior surface thereof) that protrudes into or is fitted within an internal circumferential or annular groove or recess 336 within the sleeve head 304 and is configured to enable cooperative axial movement of the actuator head 306 and the sleeve head 304, as well as the actuator member 332 and the outer sleeve 302 respectively attached thereto, while further enabling the rotational movement of the actuator head 306 relative to the sleeve head 304. Similarly, the engagement head 308 can include a head base 338 (disposed at a proximal end of the threaded member 312) that protrudes into or is fitted or received within an internal circumferential or annular recess 340 within an actuator base 342 and is configured to enable cooperative axial movement of the engagement head 308 and the actuator head 306, as well as the engagement member 330 and the actuator member 332 respectively attached thereto, while further enabling the rotational movement of the engagement head 308 relative to the actuator head 306.

In alternate examples, the sleeve head, actuator head, and the engagement head can have different features or a different combination of features that similarly limit or restrict axial movement relative to each other, while enabling rotation of each of the actuator head and the engagement head relative to the sleeve head. For example, an interior surface of the sleeve head can include one or more annular projections received within one or more circumferential or annular grooves or recesses in an exterior surface of the actuator head. In another example, an interior surface of the actuator head can include one or more annular projections received within one or more circumferential or annular grooves or recesses in an exterior surface of the engagement head.

Also illustrated in FIGS. 6A-7, the engagement member 330 can be disposed within and rotatable within a lumen of the actuator member 332, while the actuator member 332 can be disposed within and rotatable within a lumen the outer sleeve 302. In other words, in some examples of the actuation assembly 300, the outer sleeve 302 comprises an outermost fixed layer (configured to be non-rotatable when coupled to the frame), the actuator member 332 comprises a coaxial intermediate rotatable layer (configured to be rotatable when coupled to the frame), and the engagement member 330 comprises a coaxial innermost rotatable layer (configured to be rotatable when coupled to the frame).

It will be appreciated that the actuation assembly 300 can extend through a shaft or lumen of a delivery apparatus (such as through the outer shaft 206 of the delivery apparatus 200). As mentioned above, the delivery apparatus can include a plurality of actuator assemblies 300, each of which can be releasably coupled to a respective actuator or threaded rod 126 of the prosthetic valve and can extend through the shaft of the delivery apparatus. Each of the three layers (the outer sleeve 302, the actuator member 332, and the engagement member 330) can be configured to move together axially through the delivery apparatus (as described above), and rotation of each of the actuator member 332 and the engagement member 330 can be individually controlled by a knob or other actuator on a handle of the delivery apparatus. Each of the actuator member 332 and the engagement member 330 can be configured as a torque-transmitting member.

Referring to FIGS. 3A and 3B, to couple an actuation assembly 300 to the frame of a prosthetic valve, the actuation assembly 300 can be axially advanced so that the sleeve head 304 is aligned with the proximal end 110 of the proximal post 124a. As illustrated in FIG. 6A, the sleeve head 304 can be brought into contact with the proximal post 124a. The threaded engagement member 312 of the engagement head 308 can then be aligned with an opening of the threaded socket 171, and the engagement member 330 can be rotated in a first direction (for example, clockwise) relative to the actuator member 332 to threadedly engage the threaded engagement member 312 with the threaded socket 171 and to cause axial advancement of the threaded engagement member 312 into the threaded socket 171 in a proximal to distal direction.

As the threaded engagement member 312 is advanced into the socket 171 (illustrated in FIGS. 6B and 7), the pair of support extensions 310 can be simultaneously advanced over the pair of arms 180 (disposed at the proximal end 110 of the proximal post 124a), thereby bracing the pair of arms 180 between the support extensions 310 and bringing the proximal face 182a of the proximal post 124a to abut the interior surface 326 of the sleeve head 304 (illustrated in FIG. 3C). Additionally, as the threaded engagement member 312 is advanced into the threaded socket 171, the socket 314 of the actuator head 306 can be simultaneously advanced over the first raised wall 173a and the second raised wall 174a of the head 131a, which brings the facets 318 on the interior of the socket 314 into engagement with the complementary facets 175a on the exterior surface of the second raised wall 174a.

Advancement of the sleeve head 304, the actuator head 306, and the engagement head 308 can be stopped or limited by (i) contact between the proximal face 182a of the proximal post 124a and the interior surface 326 of the sleeve head 304, (ii) contact between a distal end of the first raised wall 173a and a step 344 on an interior of or at a base of the collar 316 (the socket 314), and/or (iii) contact between a distal end of the collar 316 and the first shoulder 176 formed by the base 172 of the head 131a. In some examples, a fully engaged position of the actuation assembly 300 can be achieved when one or more of the foregoing contacts is formed.

Once in the engaged position (FIGS. 6B and 7), the actuation assembly 300 is configured to enable rotation of the threaded rod 126 and corresponding radial expansion or radial collapse of a mechanically actuatable frame (such as, for example, the frame 102a) comprising the threaded rod 126. Specifically, the actuator member 332 (or the actuator member 332 and the engagement member 330) can be rotated in either of a first (for example, clockwise) direction or a second (for example, counterclockwise) direction to cause rotation of (to apply torque to) the actuator or threaded rod 126. As discussed above with reference to FIG. 1B, rotation of the actuator 126 relative to the proximal post 124a and the distal post (for example, the distal post 122) of a frame in the first direction can cause the proximal and distal posts to move toward one another and decrease a gap (G) therebetween, thereby causing radial expansion of the frame. Further, rotation of the actuator 126 relative to the proximal post 124a and the distal post 122 in the second direction can cause the proximal and distal posts to move away from one another and increase a gap (G) therebetween, thereby causing radial compression of the frame.

In some examples, the respective engagements of the sleeve head 304, the actuator head 306, and the engagement head 308 with the distal end 110 of the proximal post 124a and the head 131a described above can prevent or limit unwanted movement and/or rotation of the prosthetic valve during radial expansion and/or radial compression thereof, thereby decreasing unwanted movement of the prosthetic valve (such as, for example, after placement or positioning within the native valve). For example, the fitting or bracing of the pair of arms 180 at the proximal end 110 of the proximal post 124a between the support extensions 310, particularly in examples where the support extensions have a curved shape (as in FIGS. 5A and 5B) corresponding to a curved shape of the proximal post (as in FIG. 4B), can limit unwanted lateral movement and/or rotational displacement of the frame during actuation (such as, for example, unwanted lateral movement and/or rotational displacement along Y and Z axes, wherein the Y and Z axes are perpendicular to the longitudinal axis of the actuation assembly 300). Further, the engagement of the support extensions 310 with the frame 102a in this manner can counter-act rotational forces applied to the frame 102a by the actuators 126 during for example, expansion of the frame. In the absence of a counterforce acting against such rotational forces, the frame can tend to “jerk” or rock in the direction of rotation of the rods when they are actuated to expand the frame. The illustrated configuration is advantageous in that the sleeve head 304, when engaging the proximal posts 124a of the frame 102a, can prevent or mitigate such jerking or rocking motion of the frame 102a when the frame is radially expanded.

In another example, engagement (contact) between the facets 318 on the interior of the socket 314 and the complementary facets 175a on the exterior surface of the second raised wall 174a, as well as the threaded engagement between threaded engagement member 312 and the threaded socket 171, can stably maintain the engagement or coupling between the head 131a and the socket 314 during rotation of the actuator 126 to limit unwanted lateral movement and/or rotational displacement of the frame during actuation that might otherwise result from disengagement or partial disengagement between the actuator and the head of the threaded member. In yet another example, the threaded engagement between threaded engagement member 312 and the threaded socket 171 can maintain the engaged position of the support extensions 310 with the pair of arms 180 and limit unwanted axial movement therebetween (such as, for example, unwanted axial movement the support extensions 310 relative to the pair of arms 180).

In an alternative example, the actuation assembly 300 can omit the outer sleeve sub-assembly 301 and instead rely on the threaded connection between the engagement head 308 and the head 131a of the actuator 126 to prevent or minimize movement of the frame 102a relative to the actuation assembly while the actuator sub-assembly 303 is actuated to radially expand the frame.

Additionally, it will be appreciated that, in some examples, a delivery apparatus can include the same number of actuator assemblies 300 as the number of actuators 126 included in the frame. For example, the frame 102a shown in FIGS. 1A and 1B includes six actuators 126, and therefore a delivery apparatus configured for actuation of the frame 102a can include six actuator assemblies 300. In some examples, alternating ones of the actuators 126 (a first set) can be configured to cause radial expansion when rotated in a clockwise direction, while the remaining actuators 126 (a second set) can be configured to cause radial expansion when rotated in a counterclockwise direction (and vice versa for radial compression of the frame 102a). Similarly, a first set of actuation members 332 can be rotated clockwise and a second set of actuation members 332 can be rotated counter-clockwise to rotate the actuators 126 in the same directions for radial expansion of the frame 102a (and vice versa for radial compression of the frame). For example, if the prosthetic valve includes six actuators 126, the delivery apparatus can include a first set of three actuator assemblies 300 with three actuation members 332 that are rotated clockwise to produce radial expansion of the prosthetic valve and a second set of three actuator assemblies 300 with three actuation members 332 that are rotated counter-clockwise to produce radial expansion of the prosthetic valve. The foregoing example may further enable increased stability of the frame during radial expansion and compression, as torque applied to the first set of threaded members in the clockwise direction can be balanced by the counter-torque applied the second set of threaded members (or vice versa). In other examples, each of the threaded members can be configured for rotation or driving in a common direction for actuation (for example, clockwise rotation causes radial expansion of the frame 102a, while counterclockwise rotation causes radial compression of the frame 102a).

After the prosthetic valve is radially expanded at a desired implantation site by actuation of the actuation assemblies, the actuation assemblies 300 can be disengaged from the frame. Specifically, the engagement member 330 can be rotated in a second opposing direction (for example, counterclockwise) relative to the actuator member 332 to axially withdraw the threaded engagement member 312 from the threaded socket 171 in a distal to proximal direction. As the threaded engagement member 312 is withdrawn from the threaded socket 171, the axial movement of the threaded engagement member 312 and the engagement member 330 in the distal to proximal direction results in corresponding axial movement of the actuator head 306, the actuation member 332, the sleeve head 304, and the sleeve member 302 in the distal to proximal direction, and causes disengagement of the support extensions 310 from the pair of arms 180 at the proximal end 110 of the proximal post 124a, as well as disengagement of the facets 318 on the interior of the socket 314 and the complementary facets 175a on the exterior surface of the second raised wall 174a.

Accordingly, after the threaded engagement member 312 is fully unthreaded from the threaded socket 171 for each of the actuation assemblies 300, the delivery apparatus can be retracted relative to the implanted prosthetic valve and removed from the patient's body. Utilizing the threaded mechanism of the engagement head for detachment of the actuation assembly can enable fine control over the pace and/or degree of detachment and attachment (by for example, selectively withdrawing and advancing the threaded engagement member 312). Further, the threaded detachment mechanism can result in little or no force being applied to the frame of the prosthetic valve as the engagement head is released from the frame, thereby decreasing unwanted movement of the prosthetic valve (such as, for example, after placement or positioning within the native valve).

Turning now to FIGS. 8A-12B, another exemplary actuation assembly 400 of a delivery apparatus for releasable coupling with a frame 102b for actuation thereof is shown and described. The actuation assembly 400 can be, for example, one of the actuator assemblies 208 of the delivery apparatus 200 shown in FIG. 2. The frame 102b can be utilized in the prosthetic valve 100 shown in FIG. 1A or other prosthetic valves.

It will be appreciated that the frame 102b is similar to the frame 102a, and the descriptions above with respect to the frame 102a is applicable to the frame 102b. Notably, proximal posts 124b differ from the proximal posts 124a at the proximal end 110 of the frame 102b. Specifically, as best illustrated in FIGS. 8B and 8C, the proximal posts 124b lack any extensions or arms extending outwardly therefrom at a proximal end 110b thereof (such as, for example, arms similar to the arms 180 of the proximal posts 124a). Rather, the proximal ends 110b of the proximal posts 124b are substantially flat, and the heads 131b of actuators 126 are exposed or extend above a proximal face 182b of the proximal posts 124b. Additionally, the head 131b comprises a faceted head having a different configuration relative to the head 131a. Namely, the facets or flat portions 175b are disposed on an exterior surface of the first raised wall 173b (for example, six facets, each positioned at 60° on the exterior surface relative to others of the facets), and the second raised wall 174b has a substantially smooth, continuously curved exterior surface. Further, head 131b lacks a base (such as, for example, the base 172 of the head 131a).

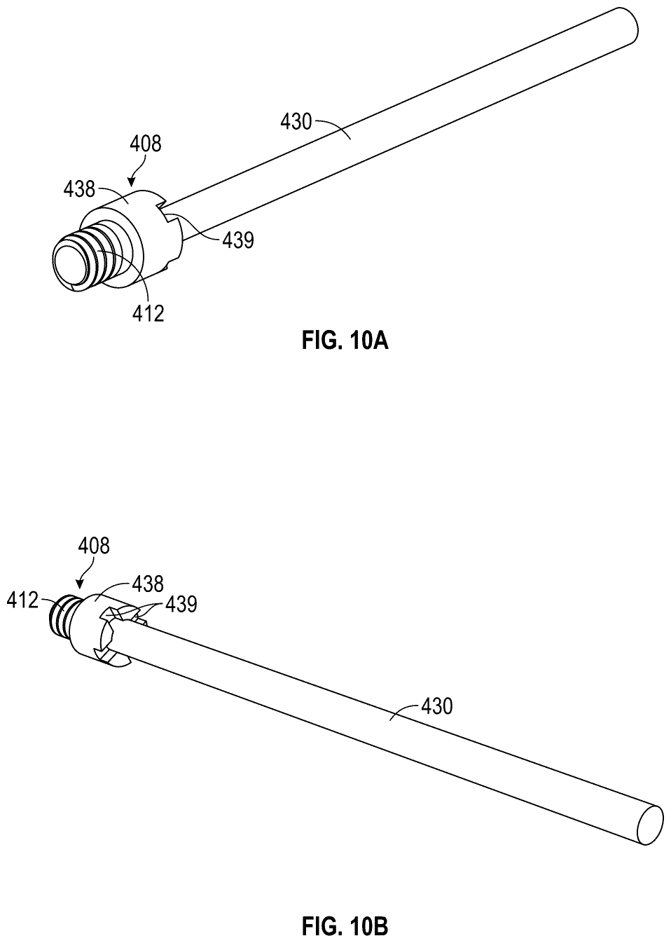

As best illustrated in FIG. 11, the actuation assembly 400 includes three concentric or coaxial sub-assemblies, including an outer sleeve sub-assembly 401, an intermediate actuator sub-assembly 403, and an inner engagement sub-assembly 405. The outer sleeve sub-assembly 401 can include an outer sleeve member or support member 402 and a sleeve head 404 connected at a distal end thereof. The actuator sub-assembly 403 can include an actuator member 432 and an actuator head 406 connected at a distal end thereof. The engagement sub-assembly 405 can include an engagement member 430 and an engagement head 408 connected at a distal end thereof.

The actuation member 432 can be coaxial with and disposed within a lumen of the outer sleeve member 402, and the engagement member 430 can be coaxial with and disposed within a lumen of the actuation member 432. Each of the actuation member 432 and the engagement member 430 can be rotatable relative to the outer sleeve member 402. Further, each of the actuation member 432 and the engagement member 430 can be rotatable relative to each other.

Also illustrated in FIG. 11, the actuation head 406 can be coaxial with and at least partially disposed within a channel of the sleeve head 404, and the engagement head 408 can be coaxial with and at least partially disposed within a channel of the actuation head 406. Each of the actuation head 406 and the engagement head 408 can be rotatable relative to the sleeve head 404. Further, each of the actuation head 406 and the engagement head 408 can be rotatable relative to each other.

Although not specifically illustrated, a proximal (opposing) end portion of each of the outer sleeve member, the actuation member, and the engagement member can be operatively coupled to a handle or other controls of the delivery apparatus to enable the physician to control rotation of the engagement member and the engagement head coupled at the distal end thereof, and to control rotation of the actuator member and the actuator head coupled at the distal end thereof. For example, the proximal end portion of the actuator member 432 of each assembly 400 can be operatively coupled to the knob 212 (FIG. 2), which is configured to rotate all of the actuator members 432 together upon rotation of the knob 212 to control radial expansion or compression of the prosthetic valve. The proximal end portion of the engagement member 430 of each assembly 400 can be operatively coupled to the knob 214 (FIG. 2), which is configured to rotate all of the engagement members 430 together to connect or disconnect the engagement heads 408 from the actuators 126 upon rotation of the knob 214.

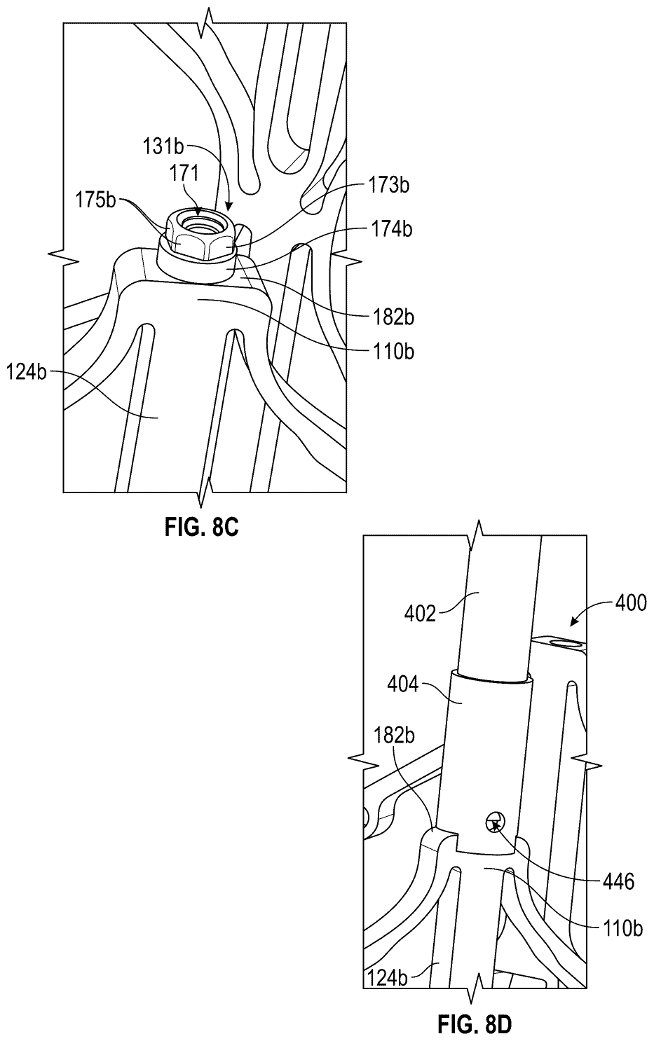

In some examples, the sleeve head 404 is can be configured to engage with the proximal end 110b of the proximal post 124b, while the actuator head 406 and the engagement head 408 are configured to engage the head 131b of the actuator 126. FIG. 9 illustrates the actuation assembly 400 in a detached state, while FIGS. 8A, 8D, and 11-12B illustrate the actuation assembly 400 releasably coupled to or engaged with the actuator 126 and the proximal post 124b.

As shown in FIG. 9, the sleeve head 404 can include a pair of support extensions 410 (a pair of prongs) distally extending at a distal end thereof, which are configured to releasably engage or releasably mate with the proximal end 110b of the proximal post 124b (for example, at interior and exterior surfaces the post 124b and at a proximal face 182b). In some examples, the sleeve and the support extensions 410 can be characterized as a stationary coupler configured to couple with a portion of the frame.

The actuator head 406 and the engagement head 408 can be configured to releasably engage or releasably mate with the head 131b of the actuators 126. In some examples, each of the actuator head 406 and the engagement head 408 can be characterized as a rotatable coupler configured to couple to a respective portion of the head 131b of the actuators 126. As the head 131b is raised relative to the proximal face 182b of the proximal post 124b, the actuation assembly 400 can have a complementary configuration in which the sleeve head 404 (including the support extensions 410) extends distally relative to the actuator head 406, which enables the support extensions 410 to extend over the head 131b and engage with side surfaces of the proximal post 124b at the proximal end 110.

As illustrated in FIGS. 9-11, the actuator head 406 can include a socket 414 formed by or defined by a collar 416, which can have a substantially smooth, continuously curved exterior surface. The engagement head 408, including a central threaded engagement member 412, is disposed within the socket 414 and can be configured to be rotatable therein. An interior surface of the socket 414 includes one or more facets or flat portions 418 evenly spaced at the interior surface thereof (such as, for example, including six facets, each positioned at 60° on the interior surface relative to others of the facets). It will be appreciated that the configuration of the facets 418 can be complementary to the configuration of the facets 175b to enable engagement therebetween (discussed in further detail below with reference to FIG. 11).

Also shown in FIGS. 9 and 11, the actuator head 406 and the threaded engagement member 412 are centrally disposed within a channel of the sleeve head 408, and the actuator head 406 can be configured to be rotatable within the channel of the sleeve head 408. Further, the sleeve head 404 can include a cylindrical body 422 and the pair of support extensions 410 can extend from a distal end of the cylindrical body 422. In other examples the cylindrical body 422 can have a different shape, such as having a cuboid, a hexagonal prism shape or other shape. The proximal end of the cylindrical body 422 can be fixedly attached to a distal end the outer sleeve 402.

As described above, the pair of support extensions 410 can be configured to enable engagement of the sleeve head 404 with a portion of the frame 102b within a prosthetic valve, such as, for example, enabling engagement with the proximal end 110b of the proximal post 124b. In some examples, the pair of support extensions 410 can have a configuration or shape that is complementary to a configuration for shape the proximal end 110b of the proximal post 124b. For example, as illustrated in FIGS. 8C-9, the surfaces of the proximal post 124b may be generally flat, and the support extensions 410 can have a similar configuration, such as being generally flat (on at least the interior surface thereof) and having equal or similar lengths. Alternatively, similar to the support extensions 310a and 310b shown in FIGS. 5A and 5C, the support extensions 410 can be configured to engage with a proximal post having a curvature (which contributes to an overall continuous curvature or cylindrical shape of the prosthetic valve).

In either example, the pair of support extensions 410 can be configured to be inserted or slid over the proximal end 110b of the proximal post 124b (for example, slid over the proximal post 124b) such that the support extensions 41 extend over the head and the interior surfaces of the support extensions 410 are abutted to the interior and exterior surfaces of the proximal end 110b of the proximal post 124b. In some examples, the proximal end 110b of the proximal post 124b is configured to be braced between the pair of support extensions 410 and limit or restrain rotational and/or lateral movement of the sleeve head 404 relative to the proximal post 124b. Further, a proximal face 182b of the proximal post 124b is configured to abut an interior surface 426 (an abutment surface disposed between the support extensions 410) of the sleeve head 404, thereby limiting axial movement in a proximal to distal direction relative to the proximal post 124b.

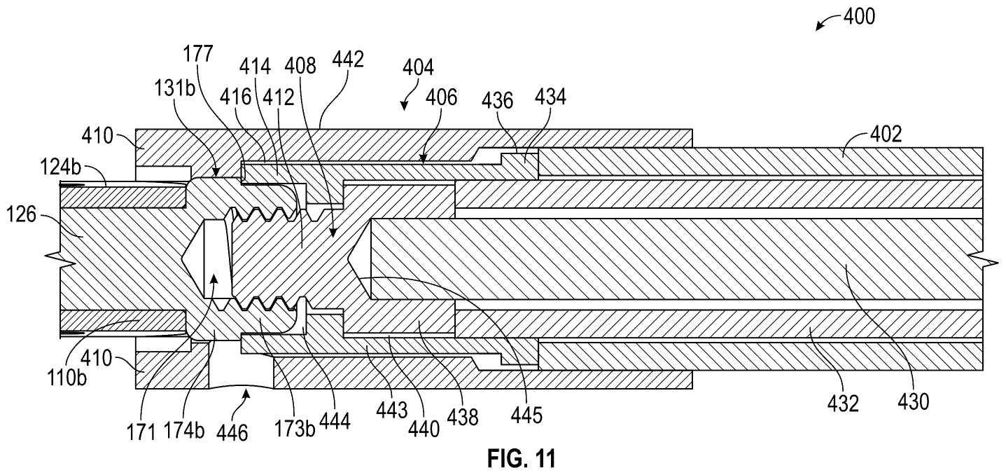

As discussed above, FIG. 9 shows the sleeve head 404, in a disengaged position relative the proximal post 124a, while FIGS. 8A, 8D and 11-12B show the sleeve head 404 (as well as the actuator head 406 and the engagement head 408) in an engaged position with the head 131b of the actuator 126 and the proximal post 124b. Specifically, in FIGS. 8A, 8D and 11-12B, the sleeve head 404 is in a position where the proximal end of the proximal post 124b is inserted and/or braced between the support arms 410 and contacts the interior surface 426 of the sleeve head 404, while the actuator head 406 engages the head 131b of the actuator 126. For illustrative purposes, the sleeve head 404 is shown as transparent in FIG. 12A.

The cross-sectional view of FIG. 11 best illustrates engagement or releasable coupling of the sleeve head 404 with the proximal end 110b of the proximal post 124b and actuator head 406 and engagement head 408 with the head 131b of the actuator 126. Specifically, FIG. 11 depicts a state where each of the sleeve head 404, the actuator head 406, and the engagement head 408 are in a fully engaged position. As discussed above, the proximal end 110 of the proximal post 124b can be braced between the pair of support extensions 410 in order limit or restrain rotational and/or lateral movement of the sleeve head 404 relative to the proximal post 124b. Further, the proximal face 182b of the proximal post 124b can be abutted to the interior surface 426 (a surface disposed between the support extensions 410), thereby limiting axial movement in the proximal to distal direction of the sleeve head 404 relative to the proximal post 124b. Additionally, the first raised wall 173b of the head 131b can be fitted within the socket 414 of the actuator head 406, such that the facets 418 within the socket 414 engage the facets 175b on the exterior surface of the first raised wall 173b. Furthermore, the threaded engagement member 412 of the engagement head 408 can be threaded into the threaded socket 171 of the head 131b.

Also illustrated in FIG. 11, the actuator head 406 can include a circumferential or annular protrusion 434 (on an exterior surface thereof) that protrudes into or is fitted within an internal circumferential or annular groove or recess 436 within the sleeve member 402 and is configured to enable cooperative axial movement of the actuator head 406 and the sleeve head 404, as well as the actuator member 432 and the outer sleeve 402 respectively attached thereto, while further enabling the rotational movement of the actuator head 406 relative to the sleeve head 404. Similarly, the engagement head 408 can include a head base 438 (disposed at a proximal end of the threaded member 412) that protrudes into or is fitted or received within an internal circumferential or annular recess 440 formed within (and between) an actuator base 443, and the actuator member 432 is configured to enable cooperative axial movement of the engagement head 408 and the actuator head 406, as well as the engagement member 430 and the actuator member 432 respectively attached thereto, while further enabling the rotational movement of the engagement head 408 relative to the actuator head 406. A distal end portion of the engagement member 430 can extend into an axially extending bore 445 formed in the engagement head base 438. The distal end portion of the engagement member 430 can be fixed to the head base 438 such as by welding, an adhesive, mechanical fasteners, and/or various other connection means.

In alternate examples, the sleeve head, actuator head, and the engagement head can have different features or a different combination of features that similarly limit or restrict axial movement relative to each other, while enabling rotation of each of the actuator head and the engagement head relative to the sleeve head. For example, an interior surface of the sleeve head can include one or more annular projections received within one or more circumferential or annular grooves or recesses in an exterior surface of the actuator head. In another example, an interior surface of the actuator head can include one or more annular projections received within one or more circumferential or annular grooves or recesses in an exterior surface of the engagement head

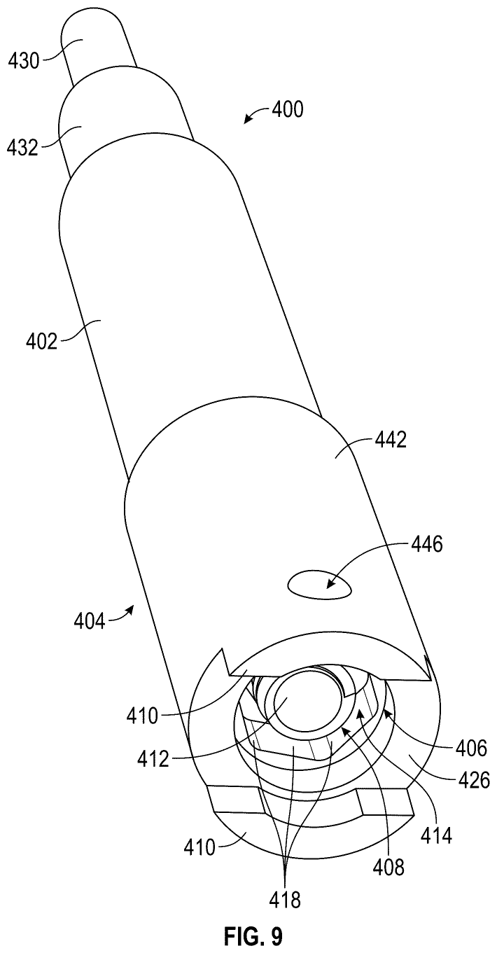

As a brief aside, detailed views of the engagement head 408 (including the threaded engagement member 412 and the head base 438) and the engagement member 430 are shown in FIGS. 10A and 10B. As can be seen therein, the head base 438 can include one or more recesses 439 at the proximal end thereof, at a connection point between the engagement head 408 and the engagement member 430. In some examples, the head base 438 can be laser welded to an outer surface of the engagement member 430, and the recesses 439 (for example, four recesses having a generally square or cuboid shape) can increase a length or surface area of the weld line between the head base 438 and the engagement member 430. In alternate examples, the recesses can be excluded from the base head and the weld line can extend along a circular path between a proximal portion of the base head and the outer surface of the engagement member. It will be appreciated that, in some examples, the head base 338 shown in FIGS. 6A-7 can include similar recesses for welding thereof to the engagement member 330.

Returning to FIG. 11, the engagement member 430 can be disposed within and rotatable within a lumen of the actuator member 432, while the actuator member 432 can be disposed within and rotatable within a lumen the outer sleeve 402. In other words, in some examples of the actuation assembly 400, the outer sleeve 402 comprises an outermost fixed layer, the actuator member 432 comprises a coaxial intermediate rotatable layer, and the engagement member 430 comprises a coaxial innermost rotatable layer.

It will be appreciated that the actuation assembly 400 can extend through a shaft or lumen of a delivery apparatus (such as through the outer shaft 206 of the delivery apparatus 200). As mentioned above, the delivery apparatus can include a plurality of actuator assemblies 400, each of which can be releasably coupled to a respective actuator 126 of the prosthetic valve and can extend through the shaft of the delivery apparatus. Each of the three layers (the outer sleeve 402, the actuator member 432, and the engagement member 430) can be configured to move together axially through the delivery apparatus (as described above), and rotation of each the actuator member 432 and the engagement member 430 can be individually controlled by a respective knob or other actuator on a handle of the delivery apparatus. Each of the actuator member 432 and the engagement member 430 can be configured as a torque-transmitting member.

Referring to FIGS. 11-12D, to couple an actuation assembly 400 to the frame of a prosthetic valve, the actuation assembly 400 can be axially advanced so that the sleeve head 404 is aligned with the proximal end 110b of the proximal post 124b. After alignment, the sleeve head 404 can be brought into contact with the proximal post 124b. The threaded engagement member 412 can then be aligned with an opening of the threaded socket 171, and the engagement member 430 can be rotated in a first direction (for example, clockwise) relative to the actuator member 432 to threadedly engage the threaded engagement member 412 and the threaded socket 171 and to cause axial advancement of the threaded engagement member 412 into the threaded socket 171 in a proximal to distal direction.

As the threaded engagement member 412 is advanced into the socket 171, the pair of support extensions 410 can be simultaneously advanced over the proximal end 110b of the proximal post 124b, thereby bracing the proximal post 124b between the support extensions 410 and bringing the proximal face 182b of the proximal post 124b to abut the interior face 426 (disposed between the support extensions 410) of the sleeve head 404 (illustrated in FIG. 11). Additionally, as the threaded engagement member 412 is advanced into the threaded socket 171, the socket 414 of the actuator head 406 can be simultaneously advanced over the first raised wall 173b, which brings the facets 418 on the interior of the socket 414 into engagement with the complementary facets 175b on the exterior surface of the first raised wall 173b.

Advancement of the sleeve head 404, the actuator head 406, and the engagement head 408 can be stopped or limited by (i) contact between the proximal face 182b of the proximal post 124b and the interior face 426 of the sleeve head 404, (ii) contact between a distal end of the first raised wall 173b and an interior surface 444 at a base of the cylindrical wall 416 (the socket 414), and/or (iii) contact between a distal end of cylindrical wall 416 and a shoulder 177 formed between the first raised wall 173b and the second raised wall 174b of the head 131b. In some examples, a fully engaged position of the actuation assembly 400 with the proximal end 110 of the proximal post 124b and the head 131b of the actuator 126 can be achieved when one or more of the foregoing contacts is formed.

As best illustrated in FIGS. 12A and 12B, the actuation assembly 400 can further include a window 446 disposed in the sleeve head 404. In some examples, the window 446 can be configured to enable visualization of contact between the head 131b and the actuator head 406 (that is, visualization of the contact between the distal end of cylindrical wall 416 and a shoulder 177 formed between the first raised wall 173b and the second raised wall 174b of the head 131b). Thus, in some examples, the window 446 can allow a physician or other person attaching the actuation assembly 400 to the frame 102b to confirm full engagement prior to insertion of the prosthetic valve and the actuator assemblies into a capsule of the delivery apparatus (even in a condition where the head 131b of the actuator 126 is covered by the sleeve head 404).

Once in the engaged position (FIGS. 11-12B), the actuation assembly 400 is configured to enable rotation of the actuator 126 and corresponding radial expansion or radial collapse of a mechanically actuatable frame (such as, for example, the frame 102b) comprising the actuator 126. Specifically, the actuator member 432 (or the actuator member 432 and the engagement member 430) can be rotated in either of a first (for example, clockwise) direction or a second (for example, counterclockwise) direction to cause rotation of (to apply torque to) the actuator 126. As discussed above, rotation of the actuator 126 relative to the proximal post 124b and the distal post 122 of the frame 102b in the first direction can cause the proximal and distal posts to move toward one another and decrease a gap (G) therebetween, thereby causing radial expansion of the frame. Further, rotation of the actuator 126 relative to the proximal post 124b and the distal post 122 in the second direction can cause the proximal and distal posts to move away from one another and increase a gap (G) therebetween, thereby causing radial compression of the frame 102b.

In some examples, the respective engagements of the sleeve head 404, the actuator head 406, and the engagement head 408 with the proximal end 110 of the proximal post 124b and the head 131b described above can prevent or limit unwanted movement and/or rotation of the prosthetic valve during radial expansion and/or radial compression thereof, thereby decreasing unwanted movement of the prosthetic valve (such as, for example, after placement or positioning within the native valve). For example, the fitting or bracing of the proximal end 110 of the proximal post 124b between the support extensions 410 can limit unwanted lateral movement and/or rotational displacement of the frame during actuation (such as, for example, unwanted lateral movement and/or rotational displacement along Y and Z axes). Further, the engagement of the support extensions 410 with the frame 102b in this manner can counter-act rotational forces applied to the frame 102b by the actuators 126 during for example, expansion of the frame. In the absence of a counterforce acting against such rotational forces, the frame can tend to “jerk” or rock in the direction of rotation of the rods when they are actuated to expand the frame. The illustrated configuration is advantageous in that the sleeve head 404, when engaging the proximal posts 124b of the frame 102b, can prevent or mitigate such jerking or rocking motion of the frame 102b when the frame is radially expanded.

In another example, engagement (contact) between the facets 418 on the interior of the socket 414 and the complementary facets 175b on the exterior surface of the first raised wall 173b, as well as the threaded engagement between threaded engagement member 412 and the threaded socket 171, can stably maintain the engagement or coupling between the head 131b and the socket 414 during rotation of the actuator 126 to limit unwanted lateral movement and/or rotational displacement of the frame during actuation that might otherwise result from disengagement or partial disengagement between the actuator head and the head of the threaded member. In yet another example, the threaded engagement between threaded engagement member 412 and the threaded socket 171 can maintain the engaged position of the support extensions 410 with the proximal post 12b (at the proximal end 110) and limit unwanted axial movement therebetween (such as, for example, unwanted axial movement of the support extensions 410 relative to the proximal post 124b).

In an alternative example, the actuation assembly 400 can omit the outer sleeve sub-assembly 401 and instead rely on the threaded connection between the engagement head 408 and the head 131b of the actuator 126 to prevent or minimize movement of the frame 102b relative to the actuation assembly while the actuator sub-assembly 403 is actuated to radially expand the frame.

Additionally, it will be appreciated that, in some examples, a delivery apparatus can include a same number of actuator assemblies 400 as a number of actuators 126 included in the frame. For example, as shown in FIG. 8A, the frame 102b includes six actuators 126, and therefore a delivery apparatus for actuation of the frame 102b includes six actuator assemblies 400. It will be appreciated that the outer sleeves 402, the actuator members 432, and the engagement members 430 of the actuator assemblies 400 are shown in a cutaway view in FIG. 8A for illustrative purposes, and in implementation the sleeve, actuator, and engagement members can extend for example, through an outer shaft of a delivery apparatus to connect with a handle or control portion.

In some examples, alternating ones of the actuators 126 and the actuation members 432 (a first set) can be configured to cause radial expansion when rotated in a clockwise direction, while the remaining actuators 126 and actuation members 432 (a second set) can be configured to cause radial expansion when rotated in a counterclockwise direction (and vice versa for radial collapse or contraction of the frame 102b). The foregoing example may further enable increased stability of the frame during radial expansion and compression, as torque applied to the first set of threaded members in the clockwise direction can be balanced by the counter-torque applied the second set of threaded members (or vice versa). In other examples, each of the threaded members can be configured for rotation or driving in a common direction for actuation (for example, all threaded members rotated in a clockwise direction can cause radial expansion of the frame 102a, while counterclockwise rotation causes radial compression of the frame 102a).

After the prosthetic valve is radially expanded at a desired implantation site by actuation of the actuation assemblies, the actuation assemblies 400 can be disengaged from the frame. Specifically, the engagement member 430 can be rotated in a second opposing direction (for example, counterclockwise) relative to the actuator member 432 to axially withdraw the threaded engagement member 412 from the threaded socket 171 in a distal to proximal direction. As the threaded engagement member 412 is withdrawn from the threaded socket 171, the axial movement of the threaded engagement member 412 and the engagement member 430 in the distal to proximal direction results in corresponding axial movement of the actuation head 406, the actuation member 432, the sleeve head 404, and the sleeve member 402 in the distal to proximal direction, and causes disengagement of the support extensions 410 from the proximal end 110 of the proximal post 124b, as well as disengagement of the facets 418 on the interior of the socket 414 and the complementary facets 175b on the exterior surface of the first raised wall 173b.

Accordingly, after the threaded engagement member 412 is fully unthreaded from the threaded socket 171 for each of the actuation assemblies 400, the delivery apparatus can be withdrawn relative to the implanted prosthetic valve and removed from the patient's body. Utilizing the threaded mechanism of the engagement head for detachment of the actuation assembly can enable fine control over the pace and/or degree of detachment and attachment (by for example, selectively withdrawing and advancing the threaded engagement member 412). Further, the threaded detachment mechanism can result in little or no force being applied to the frame of the prosthetic valve as the engagement head is released from the frame, thereby decreasing unwanted movement of the prosthetic valve (such as, for example, after placement or positioning within the native valve).

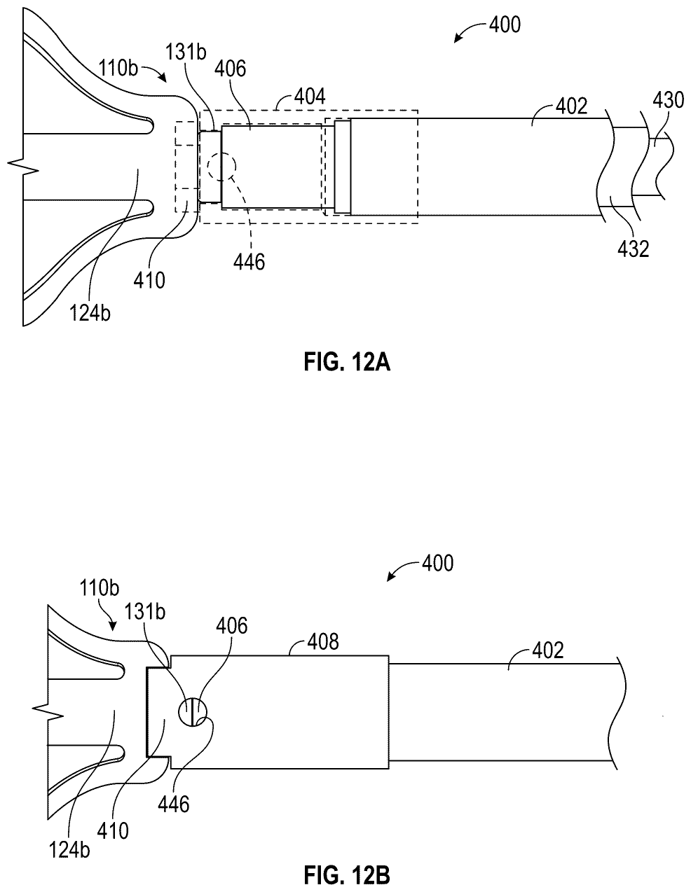

Turning now to FIGS. 13-15C, another exemplary actuation assembly 500 of a delivery apparatus for releasable coupling with a head 131c of an actuator (for example, threaded rod 126) of a frame of a prosthetic device (for example, prosthetic valve 100) for actuation thereof is shown and described. The actuation assembly 500 can be, for example, one of the actuator assemblies 208 of the delivery apparatus 200 shown in FIG. 2. The head 131c of the actuator shown in FIG. 13 can be used in lieu of the head 131a of frame 102a or head 131b of frame 102b or used with a similar actuator (for example, threaded rod) of an alternate frame for a prosthetic valve. The delivery apparatus can include a plurality of actuator assemblies 500 of the type shown in FIG. 14 for engaging respective actuators 126 of the prosthetic device.

As shown in FIG. 13, the head 131c (actuator head) can include a collar 174c (or raised wall) that is wider than a shaft of the threaded rod 126, as described above. The head 131c can further include a threaded portion 173c extending proximally from the collar 174c and comprising a plurality of external (or outer) threads 175c formed on a first surface of the head 131c. The head 131c can also include a socket 171c extending therethrough and defining a second surface (internal surface) configured to receive and interface with a portion of the actuation assembly 500, as described further below. The actuation assembly 500 can be configured to engage with the first and second surfaces of the head 131c and rotate the head 131c (and thus the threaded rod 126), thereby radially expanding and compressing the frame of which the actuator head 131c is part (as shown in FIGS. 15A-15C, as described further below).

As illustrated in the exploded view of FIG. 14, the actuation assembly 500 includes three concentric or coaxial components (or sub-assemblies), including an outer member 502 (which can also be referred to as a sleeve member or shaft), an intermediate member 504 (which can also be referred to as an engagement member or shaft), and an inner member 506 (which can also be referred to as a driver or actuation member or shaft). The intermediate member 504 can be coaxial with and disposed within a lumen of the outer member 502, and the inner member 506 can be coaxial with and disposed within a lumen of the intermediate member 504. Each of the intermediate member 504 and the inner member 506 can be rotatable relative to the outer member 502. Further, each of the intermediate member 504 and the inner member 506 can be axially movable (slidable) relative to each other. Further, in some instances, the intermediate member 504 can be rotatable relative to the inner member 506 (for example, when the inner member 506 is disengaged from the head 131c), and in other instances, the intermediate member 504 and the inner member 506 can be rotatable together (as one) (for example, when the inner member 506 and the intermediate member 504 are engaged with the head 131c).

A distal end portion of the outer member 502 can be referred to as a sleeve head 508 (or outer head) of the outer member 502. The sleeve head 508 can be configured to engage with a post of a frame, such as the proximal end 110b of the proximal post 124b. As shown in FIG. 14, the sleeve head 508 can include a pair of support extensions 510 (for example, a pair of prongs) distally extending at a distal end thereof, which are configured to releasably engage or releasably mate with the post of the frame (for example, the proximal end 110b of the proximal post 124b, such as at interior and exterior surfaces the post 124b and at a proximal face 182b). In some examples, the outer member 502 and the support extensions 510 can be characterized as a stationary coupler configured to couple with a portion of the frame. The support extensions 510 can be configured to prevent rotation of the prosthetic valve frame while rotating the actuator 126 with the intermediate member 504 and inner member 506.

The intermediate member 504 can comprise an engagement head 512 (or intermediate head) disposed at a distal end portion thereof. The engagement head 512 can comprise a threaded socket 514 comprising a plurality of internal threads 516 configured to engage and mate with the threads 175c of the actuator head 131c (as shown in FIGS. 15A and 15B). The intermediate member 504 can be configured to attach and detach to the actuator head 131c, and also actuate (for example, rotate) the actuator 126 in both clockwise and counterclockwise directions (along with the inner member 506, as described below).

The inner member 506 can comprise an actuation head 518 disposed at a distal end portion thereof. The actuation head 518 can be shaped to mate with and fit within the socket 171c of the actuator head 131c. In some instances, as shown in FIGS. 13-15C, the socket 171c and the actuation head 518 can have complementary square-shaped cross-sections. However, in some examples, the complementary cross-sections of the socket 171c and the actuation head 518 can have a different shape, such as rectangular, circular, triangular, hexagonal, and the like. As described further below, the actuation head 518 can be configured to engage with the socket 171c and prevent relative rotation between the actuator head 131c and the intermediate member 504 when the actuation head 518 is so engaged within the socket 171c.

FIGS. 15A-15C illustrate a method for coupling the actuation assembly 500 to a frame of a prosthetic valve and actuating the actuators 126 to radially expand (and/or compress) the frame. In some examples, the actuation assembly 500 can be axially advanced so that the sleeve head 508 is aligned with a proximal end of a post of the prosthetic valve frame (for example, the proximal end 110b of the proximal post 124b, as described above with reference to FIGS. 11-12D).

The intermediate member 504 can be rotated (for example, clockwise) to engage the internal threads 516 with the external threads 175c of the actuator head 131c, thereby coupling (removably coupling) the intermediate member 504 to the actuator head 131c of the actuator 126 (FIG. 15A). The inner member 506 can then be moved axially (distally) toward and into the socket 171c of the actuator head 131c (FIG. 15A), thereby locking relative rotation between the actuator head 131c and the intermediate member 504. In some examples, the socket 171c can comprise an end wall 172c that serves as a stop for a distal end of the actuation head 518, thereby preventing the inner member 506 from moving further in the distal direction. The sleeve head 508 of the outer member 502 can then be moved axially over the engagement head 512 and the actuator head 131c so that the support extensions 510 hold the frame in place (for example, as described above to prevent counter-rotation of the frame). The configuration shown in FIG. 15A can be referred to as an engaged position of the actuation assembly 500.

In some examples, as shown in FIGS. 15A-15C, the engagement head 512 can have a radially inwardly protruding annular wall 520 defining a central aperture through which the actuation head 518 extends. The actuation head 518 can have a width that is smaller than the diameter of the adjacent portion of the inner member 506 such that an annular shoulder 522 is defined between the actuation head 518 and the adjacent portion of the inner member 506. When the actuation head 518 is inserted into the socket 171c (FIG. 15A), the shoulder 522 can abut the adjacent surface of the wall 520. The engagement of the shoulder 522 with the wall 520 can assist in preventing the intermediate member 504 from rotating relative to the actuator 126 and the inner member 506 in a direction that would disconnect the intermediate member 504 from the actuator 126 before the user intends to perform the step of disconnecting the actuation assembly from the prosthetic device.

Once in the engaged position (FIG. 15A), the actuator 126 can be rotated (actuated) by rotating the intermediate member 504 and the inner member 506 together. For example, the actuator 126 can be rotated both clockwise and counterclockwise by rotating the intermediate member 504 and the inner member 506 together, thereby radially expanding and compressing the frame (similar to as described above for the other actuator assemblies 300 and 400).

After achieving the desired radial expansion of the frame in which the threaded rods are arranged (for example, after expanding the prosthetic device within a patient's body), the actuation assembly 500 can be detached from the actuator head 131c of the actuator 126. To enable detachment, the inner member 506 can be pulled axially away from (proximally) and out of the socket 171c of the actuator head 131c (FIG. 15B). Once the actuation head 518 is removed from the socket 171c, the intermediate member 504 can be rotated (for example, counterclockwise) to disengage the internal threads 516 from the external threads 175c of the actuator head 131c until the intermediate member 504 is fully detached from the actuator head 131c (FIG. 15C). The outer member 502 can also be moved axially away from the frame and the actuation assembly 500 can be moved away from the prosthetic valve (FIG. 15C). The configuration shown in FIG. 15C can be referred to as disengaged position of the actuation assembly 500.

It will be appreciated that, in some examples, a delivery apparatus can include a same number of actuator assemblies 500 as a number of actuators 126 included in the frame. For example, if the frame includes six threaded members (for example, as shown in FIG. 8A), a delivery apparatus for actuation of the frame can include six actuator assemblies 500.

After the intermediate member 504 is fully unthreaded from the threaded portion 173c for each of the actuation assemblies 500, the delivery apparatus can be withdrawn relative to the implanted prosthetic valve and removed from the patient's body.

In some examples of the actuation assembly 500, the outer member 502 can be omitted.

It will be appreciated that each actuation assembly 500 can extend through a shaft or lumen of a delivery apparatus (such as through the outer shaft 206 of the delivery apparatus 200). As mentioned above, the delivery apparatus can include a plurality of actuator assemblies 500, each of which can be releasably coupled to a respective actuator 126 of the prosthetic valve and can extend through the shaft of the delivery apparatus. The three layers (the outer member 502, the intermediate member 504, and the inner member 506) of each actuation assembly 500 can be configured to move together axially through the delivery apparatus (as described above), and rotation of the intermediate member 504 and axial translation of the inner member 506 can be individually controlled by a respective knob or other actuator on a handle of the delivery apparatus. Further, rotation of the intermediate member 504 and the inner member 506 together can be controlled by another knob or actuator of the handle of the delivery apparatus (or, alternatively, by the same knob that rotates the intermediate member 504 individually to connect to the actuator head 131c).

Examples of delivery apparatuses having handles with various knobs or other types of actuators for controlling rotation or axial movement of various components of actuation assemblies are disclosed in U.S. Provisional Application Nos. 63/282,463, filed Nov. 23, 2021, 63/292,285, filed Dec. 21, 2021, 63/322,294, filed Mar. 22, 2022, and 63/322,974, filed Mar. 23, 2022, which are incorporated herein by reference. Any of the actuation assemblies disclosed herein (for example, actuation assemblies 300, 400, 500) can be incorporated in a delivery apparatus disclosed in any of these prior-filed applications.

In view of the many possible examples to which the principles of the disclosed technology may be applied, it should be recognized that the illustrated examples are only exemplary and should not be taken as limiting the scope of the disclosed technology.

For example, it will be appreciated that each of the faceted-sockets of the actuator heads disclosed herein comprises a first rotatable engagement mechanism and each of the threaded engagement members of the engagement heads disclosed herein comprises a second rotatable engagement mechanism configured to be threaded into the threaded socket of a head. Having first and second rotatable engagement mechanisms (in combination with the sleeve head which stably engages a portion of the frame) enables the functions discussed above related to limiting unwanted movement and/or rotation of the prosthetic valve during radial expansion, radial compression, and/or release of the actuation assembly from the frame after radial expansion.

Such features can be enabled with other configurations for the first and second rotatable engagement mechanisms and fall within the scope of the present disclosure. For example, in alternate implementations, an exterior surface of a head of the threaded member or actuation member may be threaded and an interior surface of the socket in the actuation head can be threaded and configured to engage with the exterior surface of the bolt. Further, a socket within the head of the threaded member can be faceted (for example, having a hex configuration) and the engagement member disposed within the socket of the actuation head can have a complementary configuration (for example, a hexagonal prism) for engagement of the socket within the bolt. In other words, the first and second rotational engagement mechanisms can have an inverse configuration relative to the configurations shown and described with respect to the actuator assemblies 300 and 400 and remain within the scope of the disclosure, such as the configuration of the actuator assemblies 500 shown in FIGS. 13-15C.

### Delivery Techniques

Once a prosthetic valve (for example, prosthetic valve 100) is connected to a delivery apparatus (for example, delivery apparatus 200) by releasably coupling each actuator 126 to an actuation assembly (for example, actuation assembly 300, 400, 500) of the delivery apparatus, as described above, the prosthetic valve can be placed in a radially compressed state for delivery into a patient's body. The prosthetic valve can be radially compressed, for example, by actuating the actuation members (for example, actuation members 322 or 432, 506). Optionally, the compressed prosthetic valve can be loaded into a delivery capsule (for example, capsule 216) of the delivery apparatus. Thereafter, the delivery apparatus containing the prosthetic valve can be inserted into the patient's vasculature and advanced to the desired implantation site (for example, one of the native heart valves). Various delivery techniques for delivering the prosthetic valve to various implantation sites are described below.

Once the prosthetic valve is positioned at the desired implantation site, the prosthetic valve can be deployed from the delivery capsule (if the delivery apparatus includes a delivery capsule) and the prosthetic valve can be radially expanded by actuating the actuation members (for example, actuation members 306, 406, 506) into contact with the native anatomy or a previously implanted prosthetic device (for example, a docking device or previously implanted prosthetic valve). If the prosthetic valve is not precisely positioned at the desired location, the actuation members can be actuated to at least partially re-compress the prosthetic valve. Thereafter, the prosthetic valve can be completely removed from the patient or re-positioned and re-expanded at the implantation site. The delivery apparatus can then be disconnected from the prosthetic valve by rotating the engagement members (for example, engagement members 330, 430, 504) as previously described.

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above described examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An actuation assembly configured for actuation of a prosthetic valve comprising a mechanically actuatable frame comprising at least one actuator member, the actuator member configured to be rotated, via rotation of a head thereof, to radially expand or compress the frame, the actuation assembly comprising: an outer sleeve member; a sleeve head disposed at a distal end of the outer sleeve member and comprising a first support extension and a second support extension; an actuation member coaxially disposed within a lumen of the outer sleeve member, the actuation member being rotatable relative to the outer sleeve member; an actuation head disposed at a distal end of the actuation member and comprising a first socket, at least a portion of the actuation head extending through at least a portion of a channel within the sleeve head, the actuation head being rotatable relative to the sleeve head; an engagement member coaxially disposed within a lumen of the actuation member, the engagement member being rotatable relative to each of the sleeve member and the actuation member; and an engagement head disposed at a distal end of the engagement member, at least a portion of the engagement head disposed within the first socket, the engagement head being rotatable relative to each of the sleeve head and the actuation head; wherein the actuation assembly is configured to be transitioned between an engaged position and a disengaged position with the frame.

Example 2. The actuation assembly of any example disclosed herein, particularly example 1, wherein the actuation assembly is configured such that, in the engaged position, the first support extension and the second support extension engage opposing exterior and interior surfaces, respectively, of an end portion of the frame.

Example 3. The actuation assembly of any example disclosed herein, particularly example 2, wherein the end portion of the frame a curved configuration, and wherein the first support extension has a first length complementary to a length of the exterior surface of the portion of the frame and the second support extension has a second length complementary to a length of the interior surface of the portion of the frame, wherein the first length is greater than the second length.

Example 4. The actuation assembly of any example disclosed herein, particularly any of examples 1-3, wherein the sleeve head further comprises an abutment surface disposed between the first support extension and the second support extension, wherein the actuation assembly is configured such that, in the engaged position, the abutment surface contacts proximal face of the proximal post.

Example 5. The actuation assembly of any example disclosed herein, particularly any of examples 1-4, wherein the actuation assembly is configured such that, in the engaged position, the sleeve head and the sleeve member are non-rotatable relative to the frame.

Example 6. The actuation assembly of any example disclosed herein, particularly any of examples 1-5, wherein the actuation assembly is configured such that, in the engaged position, the sleeve head and the sleeve member limit lateral and rotational displacement of the frame during rotation of one or more of the actuation head or the engagement head.

Example 7. The actuation assembly of any example disclosed herein, particularly any of examples 1-6, wherein an interior surface of the first socket of the actuation head comprises one or more first facets, the one or more first facets having a complementary configuration to one or more second facets on an exterior surface of the head of the actuator member.

Example 8. The actuation assembly of any example disclosed herein, particularly example 7, wherein the actuation assembly is configured such that, in the engaged position, the one or more first facets of the first socket engage the one or more second facets of the head and enable, via rotation of the actuation member and the actuation head relative to the sleeve head, transmission of torque to the head of the actuator member and rotation of the actuator member.

Example 9. The actuation assembly of any example disclosed herein, particularly any of examples 1-8, wherein an exterior surface of the actuation head comprise an annular protrusion that is received within a first annular recess in an interior surface of the sleeve head, the annular protrusion configured to enable rotational movement of the actuator head relative to the sleeve head and limit axial movement of the actuator head relative to the sleeve head.

Example 10. The actuation assembly of any example disclosed herein, particularly any of examples 1-9, wherein the engagement head comprises a threaded member, wherein the engagement head is configured for rotational movement to cause, via transmission of torque from the engagement member to the engagement head, threaded engagement of the threaded member with a second socket within the head of the actuator member; and wherein the engagement head is rotatable relative to the actuation head and the sleeve when the engagement head is rotated to cause the threaded member to engage the second socket.

Example 11. The actuation assembly of any example disclosed herein, particularly example 10, wherein the engagement head further comprises a head base, the head base having a larger diameter than the threaded member, and wherein the head base is received within a second annular recess in an interior surface of the actuation head, the head base configured to enable rotational movement of the engagement head relative to the actuator head and limit axial movement of the engagement head relative to the actuator head.

Example 12. The actuation assembly of any example disclosed herein, particularly any of examples 10 or 11, wherein the sleeve head, the actuation head, and the engagement head are configured for cooperative axial movement; and wherein the actuation assembly is configured such that rotation of the threaded member in a first direction results in the threaded engagement between the threaded member and the second socket, and proximal to distal axial movement of the sleeve head, the actuator head, and the engagement head.

Example 13. The actuation assembly of any example disclosed herein, particularly example 12, wherein the actuation assembly is further configured such that rotation of the threaded member in a second direction results in threaded disengagement between the threaded member and the second socket, and distal to proximal axial movement of the sleeve head, the actuator head, and the engagement head.

Example 14. The actuation assembly of any example disclosed herein, particularly any of examples 10-13, wherein the actuation assembly is configured such that, in the engaged position, the threaded engagement of the threaded member and the second socket maintains engagement of the first support extension and the second support extension with the end portion of the frame, and further maintains engagement of the first socket of the actuation head with the head of the actuator member.

Example 15. The actuation assembly of any example disclosed herein, particularly any of examples 2-14, wherein a distal end of the actuation head and a distal end of the engagement head are distal relative to distal ends of the first support extension and the second support extension, and wherein the first support extension and the second support extension are configured to respectively engage with an interior surface and an exterior surface of a pair of arms of the end portion of the frame, wherein the head of the actuation member is disposed between a first arm and a second arm of the pair of arms.

Example 16. The actuation assembly of any example disclosed herein, particularly any of examples 2-14, wherein a distal end of the actuation head and a distal end of the engagement head are proximal relative to distal ends of the first support extension and the second support extension, and wherein the first support extension and the second support extension are configured to respectively engage with an interior surface and an exterior surface of a proximal end of the end portion of the frame, wherein the head of the threaded actuator member extends outwardly from a proximal face of the end portion of the frame.

Example 17. The actuation assembly of any example disclosed herein, particularly example 16, wherein the sleeve head comprises a window in a side surface thereof, the window configured to enable visualization of contact between the actuation head and the head of the actuator member.

Example 18. An actuation assembly configured for actuation of a prosthetic valve comprising a mechanically actuatable frame, the mechanically actuatable frame comprising a proximal post, a distal post, and an actuator member, the actuator member configured to be rotated, via rotation of a head thereof, to adjust a distance between the proximal post and the distal post, the actuation assembly comprising: an outer sleeve member; a sleeve head disposed at a distal end of the outer sleeve member and comprising a stationary coupler configured to releasably couple with a portion of the proximal post; an actuation member coaxially disposed within a lumen of the outer sleeve member, the actuation member being rotatable relative to the sleeve member; an actuation head disposed at a distal end of the actuation member and comprising a first rotatable coupler configured to releasably couple with a first portion of the head of the actuator member, the actuation head being rotatable relative to the sleeve head; an engagement member coaxially disposed within a lumen of the actuation member, the engagement member being rotatable relative to each of the sleeve member and the actuation member; and an engagement head disposed at a distal end of the engagement member and comprising a second rotatable coupler configured to releasably couple with a second portion of the head of the actuator member, the engagement head being rotatable relative to each of the sleeve head and the actuation head.

Example 19. The actuation assembly of any example disclosed herein, particularly example 18, wherein the stationary coupler comprises a pair distally extending prongs configured to be slid over an interior surface and an exterior surface of a proximal end of the proximal post, and wherein the pair of distally extending prongs are further configured to brace the interior surface and exterior surface of the proximal end of the proximal post during rotation of one or more of the actuation head or the engagement head.

Example 20. The actuation assembly of any example disclosed herein, particularly any of examples 18 or 19, wherein the stationary coupler is configured to limit one or more of rotation or displacement of the mechanically actuatable frame during rotation of one or more of the actuation head or the engagement head.

Example 21. The actuation assembly of any example disclosed herein, particularly any of examples 18-20, wherein the first rotatable coupler comprises a socket having one or more engageable structures on an interior surface thereof, the one or more engageable structures having a complementary configuration to one or more structural features on an exterior surface of the head of the actuator member and configured to be engaged therewith; and wherein the actuation member and the actuation head are configured to, when the one or more engageable structures are engaged with the one or more structural features on the exterior surface of the head of the actuator member, transmit torque to the head of the actuator member to drive rotation of the actuator member in a first direction for radial expansion of the frame.

Example 22. The actuation assembly of any example disclosed herein, particularly example 21, wherein the actuation member and the actuation head are further configured to, when the one or more engageable structures are engaged with the one or more structural features on the exterior surface of the head of the actuator member, transmit torque to the head of the actuator member to drive rotation of the actuator member in a second direction for radial collapse of the frame.

Example 23. The actuation assembly of any example disclosed herein, particularly any of examples 18-22, wherein the second rotatable coupler comprises threaded member configured to be threaded into a threaded socket of the head of the actuator member, and wherein the engagement member is configured to transmit torque to the engagement head to drive rotation of the threaded member in a first direction for threading into the threaded socket.

Example 24. The actuation assembly of any example disclosed herein, particularly example 23, wherein the sleeve head, the actuator head, and the engagement head are coupled together such that axial movement of the sleeve head, the actuator head, and the engagement head relative to each other is restricted, and such that the threading of the threaded member into the threaded socket results in: axial movement of the engagement head, the actuator head, and the sleeve head in a proximal to distal direction; coupling of the stationary coupler with the portion of the proximal post; and coupling of the first rotatable coupler with the first portion of the head of the actuator member.

Example 25. The actuation assembly of any example disclosed herein, particularly example 24, wherein the sleeve head, the actuator head, and the engagement head are configured such that threaded engagement between the threaded member and the threaded socket retains, during rotation of the actuator head, the coupling of the stationary coupler with the portion of the proximal post and the coupling of the first rotatable coupler with the first portion of the head of the actuator member.

Example 26. The actuation assembly of any example disclosed herein, particularly any of examples 24 or 25, wherein the engagement member is further configured to transmit torque to the engagement head to drive rotation of the threaded member in a second direction for unthreading the threaded member from the threaded socket; and wherein the sleeve head, the actuator head, and the engagement head are further configured such that the unthreading of the threaded member from the threaded socket results in: axial movement of the engagement head, the actuator head, and the sleeve head in a distal to proximal direction; uncoupling of the stationary coupler from the portion of the proximal post; and uncoupling of the first rotatable coupler from the first portion of the head of the actuator member.

Example 27. A system comprising: a prosthetic valve comprising a mechanically actuatable frame, the mechanically actuatable frame comprising at least one actuator member, the actuator member configured to be rotated, via rotation of a head thereof, to radially expand or compress the frame; and a delivery apparatus comprising at least one actuation assembly, the actuation assembly comprising: an outer sleeve member; a sleeve head disposed at a distal end of the outer sleeve member and comprising a stationary coupler configured to releasably couple with an end portion of the frame; an actuation member coaxially disposed within a lumen of the outer sleeve member, the actuation member being rotatable relative to the sleeve member; an actuation head disposed at a distal end of the actuation member and comprising a first rotatable coupler configured to releasably couple with a first portion of the head of the actuator member, the actuation head being rotatable relative to the sleeve head; an engagement member coaxially disposed within a lumen of the actuation member, the engagement member being rotatable relative to each of the sleeve member and the actuation member; and an engagement head disposed at a distal end of the engagement member and comprising a second rotatable coupler configured to releasably couple with a second portion of the head of the actuator member, the engagement head being rotatable relative to each of the sleeve head and the actuation head.

Example 28. The system of any example disclosed herein, particularly example 27, wherein the stationary coupler comprises a pair distally extending support extensions configured to be slid over an interior surface and an exterior surface of the end portion of the frame, and wherein the pair of distally extending support extensions are further configured to brace the interior surface and exterior surface of the end portion of the frame during rotation of one or more of the actuation head or the engagement head.

Example 29. The system of any example disclosed herein, particularly example 28, wherein a distal end of the actuation head and a distal end of the engagement head are distal relative to distal ends of the pair of distally extending support extensions, and wherein the end portion of the frame comprises a pair of arms having the head of the actuator member disposed therebetween, the head being recessed relative to a proximal end of the end portion of the frame.

Example 30. The system of any example disclosed herein, particularly example 28, wherein a distal end of the actuation head and a distal end of the engagement head are proximal relative to distal ends of the pair of support extensions, and wherein the head of the actuator member extends outwardly from a proximal face of the end portion of the frame.

Example 31. The system of any example disclosed herein, particularly example 30, wherein the sleeve head comprises a window in a side surface thereof, the window configured to enable visualization of contact between the actuator head and the head of the actuator member.

Example 32. The system of any example disclosed herein, particularly any of examples 27-31, wherein the first rotatable coupler comprises a socket having one or more engageable structures on an interior surface thereof, the one or more engageable structures having a complementary configuration to one or more structural features on an exterior surface of the head of the actuator member and configured to be engaged therewith; and wherein the actuation member and the actuation head are configured to, when the one or more engageable structures are engaged with the one or more structural features of the head of the actuator member, enable transmission of torque to the head of the actuator member to drive rotation of the actuator member in a first direction for radial expansion of the mechanically actuatable frame.

Example 33. The system of any example disclosed herein, particularly example 32, wherein the actuation member and the actuation head are further configured to, when the one or more engageable structures are engaged with the one or more structural features on the exterior surface of the head of the actuator member, enable transmission of transmit torque to the head of the actuator member to drive rotation of the actuator member in a second direction for radial collapse of the mechanically actuatable frame.

Example 34. The system of any example disclosed herein, particularly any of examples 27-33, wherein the second rotatable coupler comprises threaded member configured to be threaded into a threaded socket of the head of the actuator member, and wherein the engagement member is configured to transmit torque to the engagement head to drive rotation of the threaded member in a first direction for threading into the threaded socket.

Example 35. The system of any example disclosed herein, particularly example 34, wherein the sleeve head, the actuator head, and the engagement head are coupled together such that axial movement of the sleeve head, the actuator head, and the engagement head relative to each other is restricted, and such that the threading of the threaded member into the threaded socket results in: axial movement of the engagement head, the actuator head, and the sleeve head in a proximal to distal direction; coupling of the stationary coupler with the end portion of the frame; and coupling of the first rotatable coupler with the first portion of the head of the actuator member.

Example 36. The system of any example disclosed herein, particularly any of examples 34 or 35, wherein the sleeve head, the actuator head, and the engagement head are configured such that threaded engagement between the threaded member and the threaded socket retains, during rotation of the actuator head, the coupling of the stationary coupler with the end portion of the frame and the coupling of the first rotatable coupler with the first portion of the head of the actuator member.

Example 37. The system of any example disclosed herein, particularly any of examples 34-36, wherein the engagement member is further configured to transmit torque to the engagement head to drive rotation of the threaded member in a second direction for unthreading the threaded member from the threaded socket; and wherein the sleeve head, the actuator head, and the engagement head are further configured such that the unthreading of the threaded member from the threaded socket results in: axial movement of the engagement head, the actuator head, and the sleeve head in a distal to proximal direction; uncoupling of the stationary coupler from the end portion of the frame; and uncoupling of the first rotatable coupler from the first portion of the head of the actuator member.

Example 38. The system of any example disclosed herein, particularly any of examples 27-37, wherein the frame comprises a plurality of actuator members and the delivery apparatus comprises a plurality of actuation assemblies.

Example 39. A method of using an actuation assembly of a delivery apparatus to actuate a mechanically actuatable frame of a prosthetic valve, the actuation assembly comprising an elongate member and a head portion, the elongate member comprising a sleeve member, an actuation member coaxially disposed within and rotatably within a lumen of the sleeve member, and an engagement member disposed and being rotatable within a lumen of the actuation member, the head portion comprising a sleeve head coupled to the sleeve member, an actuation head coupled to the actuation member and at least partially disposed and being rotatable within the sleeve head, and an engagement head coupled to the engagement member and at least partially disposed and being rotatable within the actuator head, the mechanically actuatable frame comprising at least one actuator member, the actuator member configured to be rotated, via rotation of a head thereof, to radially expand or compress the frame, the method comprising: aligning the head portion of the actuation assembly with an end portion of the frame; and rotating the engagement member in a first direction to cause a threaded member of the engagement head to be threadedly engaged into a threaded socket within the head of actuator member, the threaded engagement resulting in: axial movement of the engagement head, the actuation head, and the sleeve head in a proximal to distal direction; engagement of a faceted socket of the actuation head with a faceted exterior surface of the head of the actuator member; and engagement of a pair of support extensions of the sleeve head with opposing surfaces of the end portion of the frame.

Example 40. The method of any example disclosed herein, particularly example 39, further comprising radially compressing the frame.

Example 41. The method of any example disclosed herein, particularly example 40, wherein the radially compressing the frame comprises rotating the actuation member and the actuation head in a first direction to transmit torque to the head of the actuator member and thereby causing rotation of the actuator member in the first direction.

Example 42. The method of any example disclosed herein, particularly any of examples 40 or 41, further comprising loading the prosthetic valve in the compressed state into a capsule of the delivery apparatus.

Example 43. The method of any example disclosed herein, particularly any of examples 40-42, further comprising: inserting the radially compressed prosthetic valve and the delivery apparatus into a patient's vasculature; advancing the prosthetic valve to an implantation site; and radially expanding the prosthetic valve at the implantation site.

Example 44. The method of any example disclosed herein, particularly example 43, wherein radially expanding the prosthetic valve comprises rotating the actuation member to cause rotation of the faceted socket and transmit torque to the head of the actuator member, thereby resulting in rotation of the actuator member in a first direction to radially expand the frame.

Example 45. The method of any example disclosed herein, particularly example 44, further comprising, after radial expansion of the frame, rotating the engagement member in a second opposing direction to cause the threaded member of the engagement head to be unthreaded from the threaded socket within the head of the actuator member, the unthreading resulting in: axial movement of the engagement head, the actuator head, and the sleeve head in a distal to proximal direction; disengagement of the faceted socket of the actuator head from the faceted exterior surface of the head of the actuator member; and disengagement of the pair of support extensions from the interior and exterior surfaces of the end portion of the frame.

Example 46. A delivery apparatus for implanting a prosthetic valve comprising a mechanically actuatable frame, the frame comprising at least one actuator member configured to be rotated, via rotation of a head thereof, to radially expand and compress the frame, the delivery apparatus comprising: at least one actuation assembly configured to form a releasable connection the frame, the actuation assembly comprising: an outer sleeve sub-assembly comprising a sleeve head configured to engage opposing surfaces of an end portion of the frame; an actuation sub-assembly extending through the outer sleeve sub-assembly, the actuation sub-assembly comprising an actuation head configured to engage an exterior surface of the head of the actuator member such that rotation of the actuation head produces rotation of the actuator member; and an engagement sub-assembly extending through the actuator sub-assembly, the engagement sub-assembly comprising an engagement head comprising a threaded portion configured to form a releasable threaded connection with a threaded socket of the head of the actuator member.

Example 47. The delivery apparatus of any example disclosed herein, particularly example 46, wherein the sleeve head comprises a first support extension configured to engage a first surface of the opposing surfaces of the end portion of the frame, and a second support extension configured to engage a second surface of the opposing surfaces of the end portion of the frame; and wherein the first and second support extensions are configured to, when the connection between the actuation assembly and the frame is formed, brace the end portion of the frame therebetween.

Example 48. The delivery apparatus of any example disclosed herein, particularly example 47, wherein the sleeve head further comprises an abutment surface disposed between the first support extension and the second support extension, and wherein the sleeve head is configured such that, when the connection between the actuation assembly and the frame is formed, the abutment surface contacts proximal face of the end portion of the frame.

Example 49. The delivery apparatus of any example disclosed herein, particularly any of examples 46-48, wherein the actuation assembly is configured such that, when the connection between the actuation assembly and the frame is formed, the sleeve head and the sleeve member are non-rotatable relative to the frame.

Example 50. The delivery apparatus of any example disclosed herein, particularly any of examples 46-49, wherein the actuation assembly is configured such that, when the connection between the actuation assembly and the frame is formed, the sleeve head and the sleeve member limit lateral and rotational displacement of the frame during rotation of one or more of the actuation head or the engagement head.

Example 51. The delivery apparatus of any example disclosed herein, particularly any of examples 46-50, wherein the actuation head comprises a faceted socket configured to engage one or more facets on the exterior surface of the head of the actuator member.

Example 52. The delivery apparatus of any example disclosed herein, particularly any of examples 46-51, wherein the actuation assembly is configured to, when the connection between the actuation assembly and the frame is formed, enable limiting of rotation of the actuation head and the sleeve head while the engagement head is rotated for forming the threaded connection of the threaded portion and the threaded socket of the head of the actuator member.

Example 53. The delivery apparatus of any example disclosed herein, particularly any of examples 46-52, wherein the sleeve head, the actuation head, and the engagement head are configured for cooperative axial movement; and wherein the actuation assembly is configured such that rotation of the engagement head in a first direction results in forming of the releasable threaded connection of the threaded portion and the threaded socket of the head of the actuator member and proximal to distal axial movement of the sleeve head, the actuator head, and the engagement head.

Example 54. The delivery apparatus of any example disclosed herein, particularly example 53, wherein the actuation assembly is configured such that rotation of the engagement head in a second opposing direction results in releasing the threaded connection of the threaded portion and the threaded socket of the head of the actuator member and distal to proximal axial movement of the sleeve head, the actuator head, and the engagement head.

Example 55. The delivery apparatus of any example disclosed herein, particularly any of examples 46-54, wherein the actuation assembly is configured such that, when the connection between the actuation assembly and the frame is formed, the threaded connection of the threaded portion with the threaded socket of the head of the actuator member maintains engagement of the first support extension and the second support extension with the end portion of the frame, and further maintains engagement of the actuation head and the exterior surface of the head of the actuator member.

Example 56. The delivery apparatus of any example disclosed herein, particularly any of examples 46-55, in combination with the prosthetic valve, wherein the mechanically actuatable frame further comprises a proximal post and a distal post, the actuator member configured to be rotated to adjust a distance between the proximal post and the distal post and cause the radial expansion and compression of the frame.

Example 57. The delivery apparatus of any example disclosed herein, particularly example 56, wherein the actuation sub-assembly is configured to, when the connection between the actuation assembly and the frame is formed, drive rotation of the actuation head in a first direction and transmit torque to the head of the actuator member to decrease the distance between the proximal post and the distal post and cause the radial expansion of the frame.

Example 58. The delivery apparatus of any example disclosed herein, particularly example 57, wherein the actuation sub-assembly is further configured to, when the connection between the actuation assembly and the frame is formed, drive rotation of the actuation head in a second opposing direction and transmit torque to the head of the actuator member to increase the distance between the proximal post and the distal post and cause the radial compression of the frame.

Example 59. The delivery apparatus of any example disclosed herein, particularly any of examples 46-58, wherein the at least one actuation assembly comprises a plurality of actuation assemblies and the frame comprises a corresponding plurality of actuator members.

Example 60. An actuation assembly configured for actuation of a prosthetic valve comprising a mechanically actuatable frame comprising at least one actuator member, the actuator member configured to be rotated, via rotation of a head thereof, to radially expand or compress the frame, the actuation assembly comprising: an actuation member; an actuation head disposed at a distal end of the actuation member and comprising a first socket configured to releasably couple with a first portion of the head of the actuator member; an engagement member coaxially disposed within a lumen of the actuation member, the engagement member being rotatable relative to the actuation member; and an engagement head disposed at a distal end of the engagement member, at least a portion of the engagement head disposed within the first socket, the engagement head being rotatable relative to the actuation head and configured to releasably coupled with a second portion of the head of the actuator member; wherein the actuation assembly is configured to be transitioned between an engaged position and a disengaged position with the frame.

Example 61. The actuation assembly of any example disclosed herein, particularly example 60, wherein the engagement head comprises a threaded member configured to be releasably threadedly engaged with a second socket within the head of the actuator member.

Example 62. The actuation assembly of any example disclosed herein, particularly example 61, wherein the engagement head and the actuation head are associated such that they configured to be axially moved together, and wherein rotation of the engagement head in a first direction results in the threaded engagement of the threaded member into the second socket and axial movement of the engagement head and the actuation head in a proximal to distal direction to transition the actuation assembly into the engaged position with the frame.

Example 63. The actuation assembly of any example disclosed herein, particularly any of examples 60-62, wherein the first socket comprises a faceted socket having a complementary configuration to one or more facets on the exterior surface of the head of the actuator member.

Example 64. The actuation assembly of any example disclosed herein, particularly any of examples 62-63, wherein, in the engaged position of the actuation assembly, the actuation member and the actuator head transmit torque to the head of the actuator member and drive rotation of the actuator member to radially expand or compress the frame.

Example 65. The actuation assembly of any example disclosed herein, particularly example 64, wherein the actuation assembly is configured such that, in the engaged position, the threaded engagement of the threaded member and the second socket maintains engagement between the first socket and the first portion of the head of the actuator member during rotation of the actuation head.

Example 66. The actuation assembly of any example disclosed herein, particularly any of examples 60-65, further comprising: an outer sleeve member; and a sleeve head disposed at a distal end of the outer sleeve member and comprising a first support extension and a second support extension.

Example 67. The actuation assembly of any example disclosed herein, particularly example 66, wherein the actuation member is coaxially disposed within a lumen of the outer sleeve member, each of the actuation member and the engagement member being rotatable relative to the sleeve member.

Example 68. The actuation assembly of any example disclosed herein, particularly any of examples 66 or 67, wherein at least a portion of the actuation head extends through at least a portion of a channel within the sleeve head, each of the actuation head and the engagement head being rotatable relative to the sleeve head.

Example 69. The actuation assembly of any example disclosed herein, particularly any of examples 66-68, wherein, in the engaged position of the actuation assembly, the first support extension and the second support extension are configured to receive and brace a portion of the frame therebetween and limit lateral and rotational displacement of the frame during rotation of one or more of the actuation head or the engagement head.

Example 70. The actuation assembly of any example disclosed herein, particularly example 69, wherein the actuation assembly is configured such that, in the engaged position, the threaded engagement of the threaded member and the second socket maintains a position of the first support extension and the second support extension relative to the portion of the frame.

Example 71. A delivery apparatus comprising the actuation assembly of any example disclosed herein, particularly any of examples 60-70, wherein the actuation assembly comprises a plurality of actuation assemblies.

Example 72. The actuation assembly of any example disclosed herein, particularly any of examples 60-71, in combination with the prosthetic valve, wherein the mechanically actuatable frame further comprises a proximal post and a distal post, the actuator member configured to be rotated to adjust a distance between the proximal post and the distal post and produce the radial expansion and compression of the frame.

Example 73. A method of assembling a delivery apparatus configured for delivery and actuation of a prosthetic valve comprising a mechanically actuatable frame, the method comprising: assembling at least one actuation assembly, the assembling comprising: disposing an engagement member coaxially within a lumen of an actuation member, the engagement member rotatable relative the actuation member; disposing an engagement head comprising a threaded member at least partially within a faceted socket of an actuation head, wherein the engagement head is attached at a distal end of the engagement member and the actuation head is attached at a distal end of the actuation member, wherein the engagement head is rotatable relative to the actuation head, and wherein the threaded member is configured for threaded engagement with a threaded socket in a head of an actuation member of the frame, and the faceted socket is configured to engage one or more facets on an exterior of the head of the actuation member; coupling the engagement member of an actuation assembly to a first control mechanism in a handle of the delivery apparatus, the first control mechanism configured to enable rotation of the engagement member in a first direction of the engagement member for threadedly engaging the threaded member into the threaded socket, and further configured to enable rotation of the engagement member in a second direction of the engagement member for threadedly disengaging the threaded member from the threaded socket; and coupling the actuation member of the actuation assembly to a second control mechanism in the handle, the second control mechanism configured to enable rotation of the actuation member in a first direction of the actuation member to transmit torque to the head of the actuation member for radial expansion of the frame, and further configured to enable rotation of the actuation member in a second direction of the actuation member to transmit torque to the head of the actuation member for radial collapse of the frame.

Example 74. The method of any example disclosed herein, particularly example 73, wherein assembling the actuation assembly further comprises: disposing the actuation member coaxially within a lumen of an outer sleeve member, each of the actuation member and the engagement member rotatable relative to the outer sleeve member; and disposing the actuation head at least partially within a channel of a sleeve head, wherein the sleeve head comprises a pair of prongs configured to receive a brace a portion of a frame of the prosthetic valve, and wherein the actuation head and the engagement head are rotatable relative to the sleeve head.

Example 75. The method of any example disclosed herein, particularly example 74, wherein the assembling at least one actuation assembly comprises assembling a plurality of actuation assemblies; and wherein the method further comprises: coupling the engagement member of each of the actuation assemblies to the first control mechanism; and coupling the actuation member of each of the actuation assemblies to the second control mechanism.

Example 76. The method of any example disclosed herein, particularly example 74, wherein the assembling at least one actuation assembly comprises assembling a plurality of actuation assemblies; and wherein the method further comprises: coupling the engagement member of each of a first portion of the plurality of actuation assemblies to the first control mechanism; coupling the actuation member of each the first portion of the actuation assemblies to the second control; coupling the engagement member of each of a second portion of the actuation assemblies to a third control mechanism, the third control mechanism configured to enable rotation of the engagement member in the second direction of the engagement member for threadedly engaging the threaded member into the threaded socket, and further configured to enable rotation of the engagement member in the first direction of the engagement member for threadedly disengaging the threaded member from the threaded socket; and coupling the actuation member of each of the second portion of the actuation assemblies to a fourth control mechanism, the fourth control mechanism configured to enable rotation of the actuation member in the second direction of the actuation member to transmit torque to the head of the actuation member for radial expansion of the frame, and further configured to enable rotation of the actuation member in the first direction of the actuation member to transmit torque to the head of the actuation member for radial collapse of the frame.

Example 77. An actuation assembly configured for actuation of a prosthetic valve comprising a mechanically actuatable frame comprising at least one actuator member, the actuator member configured to be rotated, via rotation of a head thereof, to radially expand or compress the frame, the actuation assembly comprising: an outer sleeve sub-assembly comprising a sleeve head having a pair of arms configured to engage opposing surfaces of an end portion of the frame; an actuation sub-assembly extending coaxially through the outer sleeve sub-assembly, the actuation sub-assembly comprising a faceted actuation head configured to engage one or more facets on the head of the actuator member such that rotation of the actuation head produces rotation of the actuator member; and an engagement sub-assembly extending coaxially through the outer sleeve sub-assembly, the engagement sub-assembly comprising an engagement head comprising a threaded portion configured to form a releasable threaded connection with a complementary threaded portion of the head of the actuator member; wherein the actuation assembly is configured to be transitioned between an engaged position and a disengaged position with the frame.

Example 78. The actuation assembly of any example disclosed herein, particularly example 77, wherein the engagement sub-assembly extends coaxially through the actuation sub-assembly.

Example 79. The actuation assembly of any example disclosed herein, particularly any of examples 77 or 78, wherein the engagement head is at least partially disposed and rotatable within a channel of the actuation head.

Example 80. The actuation assembly of any example disclosed herein, particularly any of examples 77-79, wherein the engagement head comprises a threaded member configured to be releasably threadedly engaged with a threaded socket within the head of the actuator member.

Example 81. The actuation assembly of any example disclosed herein, particularly example 80, wherein the engagement head, the actuation, and the sleeve head are associated such that they configured to be axially moved together; and wherein rotation of the engagement head in a first direction results in threaded engagement of the threaded member into the threaded socket and axial movement of the engagement head, the actuation head, and the sleeve head in a proximal to distal direction to transition the actuation assembly into an engaged position with the frame, and wherein rotation of the engagement head in a second direction results in threaded disengagement of the threaded member from the threaded socket and axial movement of the engagement head, the actuation head, and the sleeve head in a distal to proximal direction to transition the actuation assembly into an engaged position with the frame.

Example 82. The actuation assembly of any example disclosed herein, particularly any of examples 77-81, wherein the actuation assembly is configured such that, in an engaged position with the frame, the threaded engagement of the threaded portion of the engagement head and the complementary threaded portion of the head of the actuator member maintains the engagement between opposing surfaces of the end portion of the frame position and the pair of arms of the sleeve head.

Example 83. The actuation assembly of any example disclosed herein, particularly any of examples 77-82, wherein the faceted actuation head comprises a faceted socket having a complementary configuration to one or more facets on the exterior surface of the head of the actuator member.

Example 84. The actuation assembly of any example disclosed herein, particularly any of examples 77-83, wherein the actuation assembly is configured such that, when in an engaged position with the frame, the outer sleeve sub-assembly limits lateral and rotational displacement of the frame during rotation of one or more of the actuation head or the engagement head.

Example 85. The actuation assembly of any example disclosed herein, particularly any of examples 77-84, wherein, in an engaged position of the actuation assembly with the frame, the actuation member and the actuator head can transmit torque to the head of the actuator member and drive rotation of the actuator member to radially expand or compress the frame.

Example 86. A delivery apparatus comprising the actuation assembly of any example disclosed herein, particularly any of examples 77-85, wherein the actuation assembly comprises a plurality of actuation assemblies.

Example 87. The actuation assembly of any example disclosed herein, particularly any of examples 77-86, in combination with the prosthetic valve, wherein the mechanically actuatable frame further comprises a proximal post and a distal post, the actuator member configured to be rotated to adjust a distance between the proximal post and the distal post and produce the radial expansion and compression of the frame.

Example 88. A prosthetic valve configured to be transitioned between a radially compressed state and a radially expanded state via an actuation assembly, the actuation assembly comprising an outer sleeve sub-assembly, an actuation sub-assembly coaxially extended through the outer sleeve sub-assembly and rotatable relative to the outer sleeve sub-assembly, and an engagement assembly coaxially extended through the actuation sub-assembly and rotatable relative to each of the actuation sub-assembly and the outer sleeve sub-assembly, the prosthetic valve comprising: a mechanically actuatable frame, a portion of the frame configured to releasably engage with a pair of support arms of a sleeve head of the sleeve sub-assembly and to be braced therebetween; an actuator member comprising a head, the actuation member configured to be rotated in a first direction to radially compress to the frame, and further configured to be rotated in a second direction to radially expand the frame; wherein the head comprises a threaded socket configured for releasable threaded coupling with a threaded member of an engagement head of the engagement sub-assembly, and a faceted outer surface configured for releasable engagement with a faceted socket of an actuation head of the actuation sub-assembly.

Example 89. The prosthetic valve of any example disclosed herein, particularly example 88, wherein the mechanically actuatable frame comprises a proximal post and a distal post having a distance therebetween, the actuation member configured to be rotated in a first direction to increase the distance between the proximal pose and the distal post to radially compress to the frame, and further configured to be rotated in a second direction to decrease the distance between the proximal post and the distal post to radially expand the frame.

Example 90. The prosthetic valve of any example disclosed herein, particularly any of examples 88 or 89, wherein the mechanically actuatable frame is configured such that the threaded coupling of the threaded socket with the threaded member results in the portion of the frame engaging with the pair of support arms.

Example 91. The prosthetic valve of any example disclosed herein, particularly any of examples 88-90, wherein the head is disposed above a proximal face of the frame.

Example 92. The prosthetic valve of any example disclosed herein, particularly any of examples 88-90, wherein the head is recessed relative to a proximal face of the frame.

Example 93. A delivery apparatus for implanting a prosthetic valve comprising a mechanically actuatable frame, the frame comprising at least one actuator member configured to be rotated, via rotation of a head thereof, to radially expand and compress the frame, the delivery apparatus comprising: at least one actuation assembly configured to be releasably coupled to the frame, the actuation assembly comprising: a first member comprising threads disposed on a distal end portion thereof that are configured to form a first releasable connection with a threaded portion of the head of the actuator member; and a second member that is coaxial with the first member and has distal end portion configured to form a second releasable connection with the head of the actuator member, wherein the first member and the second member are rotatable together to rotate the actuator member and expand the prosthetic valve.

Example 94. The delivery apparatus of any example disclosed herein, particularly example 93, wherein the actuation assembly further comprises a third member that is coaxial with and disposed around each of the first member and the second member, wherein the first member and the second member are rotatable relative to the third member.

Example 95. The delivery apparatus of any example disclosed herein, particularly either example 93 or example 94, wherein the distal end portion of the second member has a shape that is configured to mate with and fit within or around a complementary-shaped portion of the head of the actuator member such that the second releasable connection is formed.

Example 96. The delivery apparatus of any example disclosed herein, particularly any one of examples 93-95, wherein the first member extends coaxially through the second member.

Example 97. The delivery apparatus of any example disclosed herein, particularly example 96, wherein the threads of the first member are external threads that are configured to mate with internal threads of a first socket of the head of the actuator member.

Example 98. The delivery apparatus of any example disclosed herein, particularly either example 96 or example 97, wherein the distal end portion of the second member comprises a second socket defining an interior surface comprising one or more first facets, the one or more first facets having a complementary configuration to one or more second facets on an exterior surface of the head of the actuator member.

Example 99. The delivery apparatus of any example disclosed herein, particularly any one of examples 93-95, wherein the second member extends coaxially through the first member.

Example 100. The delivery apparatus of any example disclosed herein, particularly example 99, wherein the threads of the first member are internal threads disposed on a socket of the of the distal end portion of the first member that are configured to mate with external threads on a threaded portion of the head of the actuator member.

Example 101. The delivery apparatus of any example disclosed herein, particularly either example 99 or example 100, wherein the distal end portion of the second member is an actuation head having a cross-section shaped to fit within a complementary shaped socket of the head of the actuator member.

Example 102. The delivery apparatus of any example disclosed herein, particularly example 101, wherein the cross-section of the actuation head of the second member is square-shaped.

Example 103. A system comprising: a prosthetic valve comprising a mechanically actuatable frame, the mechanically actuatable frame comprising at least one actuator member, the actuator member configured to be rotated, via rotation of a head thereof, to radially expand or compress the frame; and a delivery apparatus comprising at least one actuation assembly, the actuation assembly comprising: a first member comprising a threaded socket at its distal end that is configured to engage with external threads on a threaded portion of the head of the actuator member; and a second member disposed coaxially within the first member, the second member comprising an actuation head at its distal end that is shaped to mate with and fit within a socket of the head of the actuator member, wherein the first member and the second member are rotatable together to rotate the actuator member and expand the prosthetic valve.

Example 104. The system of any example disclosed herein, particularly example 103, wherein the actuation assembly is movable between an engaged position and disengaged position with the frame, wherein in the engaged position the threaded socket of the first member is engaged with the external threads on the threaded portion of the head of the actuator member and the actuation head of the second member is disposed within and engaged with the socket of the head of the actuator member, and wherein in the engaged position the first member and the second member are rotatable together to rotate the actuator member and radially expand or compress the frame.

Example 105. The system of any example disclosed herein, particularly example 104, wherein in the disengaged position the actuation head of the second member is disposed axially away from the socket of the head of the actuator member and the threaded socket of the first member is disengaged from the external threads of the head of the actuator member.

Example 106. The system of any example disclosed herein, particularly example 105, wherein the first member is rotatable relative to the second member when the actuation head of the second member is disengaged from the socket of the head of the actuator member.

Example 107. The system of any example disclosed herein, particularly any one of examples 103-106, wherein the socket of the head of the actuator member extends through the threaded portion of the head of the actuator member such that the threads of the threaded portion are external to the socket of the head of the actuator.

Example 108. The system of any example disclosed herein, particularly any one of examples 103-107, wherein the actuation head has a cross-sectional shape that is complementary to a cross-sectional shape of the socket of the head of the actuator member.

Example 109. The system of any example disclosed herein, particularly example 108, wherein the cross-sectional shape of the actuation head is square.

Example 110. The system of any example disclosed herein, particularly any one of examples 103-109, wherein the actuation assembly further comprises an outer, third member comprising a pair of support extensions configured to engage with a post of the frame.

Example 111. The system of any example disclosed herein, particularly example 110, wherein the first member and the second member are rotatable relative to the third member.

Example 112. The system of any example disclosed herein, particularly either example 110 or example 111, wherein the second member is axially movable relative to the second member and the third member.

Example 113. The system of any example disclosed herein, particularly any one of examples 110-112, wherein the third member is axially movable relative to the first member and the second member.

Example 114. An actuation assembly configured for actuation of a prosthetic valve comprising a mechanically actuatable frame comprising at least one actuator member, the actuator member configured to be rotated, via rotation of a head thereof, to radially expand or compress the frame, the actuation assembly comprising: an outer member, wherein a distal end portion of the outer member comprises a first support extension and a second support extension; an intermediate member coaxially disposed within a lumen of the outer member, wherein a distal end portion of the intermediate member comprises a first socket configured to interface with a first surface of the head of the actuator member; and an inner member coaxially disposed within a lumen of the intermediate member, wherein a distal end portion of the inner member is configured to interface with a second surface of the head of the actuator member, and wherein the intermediate member and the inner member are rotatable relative to the outer member; wherein the actuation assembly is configured to be transitioned between an engaged position and a disengaged position with the frame.

Example 115. The actuation assembly of any example disclosed herein, particularly example 114, wherein the actuation assembly is configured such that, in the engaged position, the first support extension and the second support extension engage opposing exterior and interior surfaces, respectively, of an end portion of the frame.

Example 116. The actuation assembly of any example disclosed herein, particularly either example 114 or 115, wherein the actuation assembly is configured such that, in the engaged position, the outer member is non-rotatable relative to the frame.

Example 117. The actuation assembly of any example disclosed herein, particularly any one of examples 114-116, wherein the actuation assembly is configured such that, in the engaged position, the outer member limits lateral and rotational displacement of the frame during rotation of the intermediate member and the inner member.

Example 118. The actuation assembly of any example disclosed herein, particularly any one of examples 114-117, wherein an interior surface of the first socket of the intermediate member comprises one or more first facets, the one or more first facets having a complementary configuration to one or more second facets formed on the first surface of the head of the actuator member, the first surface being an external surface of the head of the actuator member.

Example 119. The actuation assembly of any example disclosed herein, particularly example 118, wherein the actuation assembly is configured such that, in the engaged position, the one or more first facets of the first socket engage the one or more second facets of the head and enable, via rotation of the intermediate member relative to the outer member, transmission of torque to the head of the actuator member and rotation of the actuator member.

Example 120. The actuation assembly of any example disclosed herein, particularly either example 118 or 119, wherein the actuation assembly is configured such that, in the engaged position, external threads on an exterior surface of the distal end portion of the inner member engage with complementary threads formed on the second surface of the head of the actuator member, the second surface being an internal surface of a second socket of the head of the actuator member.

Example 121. The actuation assembly of any example disclosed herein, particularly example 120, wherein actuation assembly is configured such that rotation of the inner member in a first direction results in threaded engagement between the external threads on the distal end portion of the inner member and the complementary threads of the head of the actuator member and rotation of the inner member in a second direction results in threaded disengagement between the external threads on the distal end portion of the inner member and the complementary threads of the head of the actuator member.

Example 122. The actuation assembly of any example disclosed herein, particularly any one of examples 114-117, wherein an interior surface of the first socket of the intermediate member comprises internal threads, and wherein the distal end portion of the inner member comprises an actuation head shaped to fit within a second socket extending through the head of the actuator member, the second socket defining the second surface.

Example 123. The actuation assembly of any example disclosed herein, particularly example 122, wherein the actuation assembly is configured such that, in the engaged position, the internal threads on the first socket of the intermediate member engage with complementary threads formed on the first surface of the head of the actuator member, and the actuation head is disposed within the second socket of the head of the actuator member.

Example 124. The actuation assembly of any example disclosed herein, particularly example 123, wherein the actuation assembly is configured such that, in the engaged position, the intermediate member and the inner member are rotatable together, relative to the outer member, to rotate the actuator member and radially expand or compress the frame.

Example 125. The actuation assembly of any example disclosed herein, particularly any one of examples 122-124, wherein the inner member is axially movable relative to the intermediate member, and wherein the intermediate member is rotatable relative to the inner member.

Example 126. A delivery apparatus for implanting a prosthetic valve comprising a mechanically actuatable frame, the frame comprising at least one actuator member configured to be rotated, via rotation of a head thereof, to radially expand and compress the frame, the delivery apparatus comprising: at least one actuation assembly configured to form a releasable connection the frame, the actuation assembly comprising: an outer sleeve sub-assembly comprising a sleeve head configured to engage opposing surfaces of an end portion of the frame; an actuation sub-assembly extending through the outer sleeve sub-assembly, the actuation sub-assembly comprising an actuation head comprising one or more planar surfaces configured to engage one or more complementary planar surfaces of the head of the actuator member; and an engagement sub-assembly extending through the outer sleeve sub-assembly, the engagement sub-assembly comprising an engagement head comprising a threaded portion configured to form a releasable threaded connection with a threaded portion of the head of the actuator member.

Example 127. The delivery apparatus of any example disclosed herein, particularly example 126, wherein the sleeve head comprises a first support extension configured to engage a first surface of the opposing surfaces of the end portion of the frame, and a second support extension configured to engage a second surface of the opposing surfaces of the end portion of the frame; and wherein the first and second support extensions are configured to, when the connection between the actuation assembly and the frame is formed, brace the end portion of the frame therebetween.

Example 128. The delivery apparatus of any example disclosed herein, particularly either of examples 126 or 127, wherein the engagement sub-assembly extends coaxially through the actuation sub-assembly such that at least a portion of the engagement head extends into at least a portion of the actuation head, the engagement head rotatable relative to the actuation head.

Example 129. The delivery apparatus of any example disclosed herein, particularly either of examples 126 or 127, wherein the actuation sub-assembly extends coaxially through the engagement sub-assembly such that at least a portion of the actuation head extends into at least a portion of the engagement head, the engagement head rotatable relative to the actuation head.

Example 130. A method comprising sterilizing a system or a delivery apparatus of any of the examples disclosed herein, particularly examples 1-129.

The features described herein with regard to any example can be combined with other features described in any one or more of the other examples, unless otherwise stated. For example, any one or more of the features of one actuation assembly can be combined with any one or more features of another actuation assembly. As another example, any one or more features of one delivery apparatus can be combined with any one or more features of another delivery apparatus.

In view of the many possible examples to which the principles of the disclosed invention may be applied, it should be recognized that the illustrated examples are only preferred examples of the invention and should not be taken as limiting the scope of the invention. Rather, the scope of the invention is defined by the following claims. We therefore claim as our invention all that comes within the scope and spirit of these claims.

We claim:

1. A delivery apparatus for implanting a prosthetic valve comprising a mechanically actuatable frame, the frame comprising at least one actuator member configured to be rotated, via rotation of a head thereof, to radially expand and compress the frame, the delivery apparatus comprising:
at least one actuation assembly configured to form a releasable connection the frame, the actuation assembly comprising:
an outer sleeve sub-assembly comprising a sleeve head configured to engage opposing surfaces of an end portion of the frame;
an actuation sub-assembly extending through the outer sleeve sub-assembly, the actuation sub-assembly comprising an actuation head comprising one or more planar surfaces configured to engage one or more complementary planar surfaces of the head of the actuator member; and
an engagement sub-assembly extending through the outer sleeve sub-assembly, the engagement sub-assembly comprising an engagement head comprising a threaded portion configured to form a releasable threaded connection with a threaded portion of the head of the actuator member.

2. The delivery apparatus of claim 1, wherein the sleeve head comprises a first support extension configured to engage a first surface of the opposing surfaces of the end portion of the frame, and a second support extension configured to engage a second surface of the opposing surfaces of the end portion of the frame; and
wherein the first and second support extensions are configured to, when the connection between the actuation assembly and the frame is formed, brace the end portion of the frame therebetween.

3. The delivery apparatus of claim 1, wherein the engagement sub-assembly extends coaxially through the actuation sub-assembly such that at least a portion of the engagement head extends into at least a portion of the actuation head, wherein the engagement head is rotatable relative to the actuation head.

4. The delivery apparatus of claim 1, wherein the actuation sub-assembly extends coaxially through the engagement sub-assembly such that at least a portion of the actuation head extends into at least a portion of the engagement head, wherein the engagement head is rotatable relative to the actuation head.

5. A delivery apparatus for implanting a prosthetic valve comprising a mechanically actuatable frame, the frame comprising at least one actuator member configured to be rotated, via rotation of a head thereof, to radially expand and compress the frame, the delivery apparatus comprising:
at least one actuation assembly configured to be releasably coupled to the frame, the actuation assembly comprising:
a first member comprising threads disposed on a distal end portion thereof that are configured to form a first releasable connection with a threaded portion of the head of the actuator member; and
a second member that is coaxial with the first member and has distal end portion configured to form a second releasable connection with the head of the actuator member, wherein the first member and the second member are rotatable together to rotate the actuator member and expand the prosthetic valve.

6. The delivery apparatus of claim 5, wherein the actuation assembly further comprises a third member that is coaxial with and disposed around each of the first member and the second member, wherein the first member and the second member are rotatable relative to the third member.

7. The delivery apparatus of claim 5, wherein the distal end portion of the second member has a shape that is configured to mate with and fit within or around a complementary-shaped portion of the head of the actuator member to form the second releasable connection.

8. The delivery apparatus of claim 5, wherein the first member extends coaxially through the second member.

9. The delivery apparatus of claim 8, wherein the threads of the first member are external threads that are configured to mate with internal threads of a first socket of the head of the actuator member.

10. The delivery apparatus of claim 8, wherein the distal end portion of the second member comprises a second socket defining an interior surface comprising one or more first facets, wherein the one or more first facets have a complementary configuration to one or more second facets on an exterior surface of the head of the actuator member.

11. The delivery apparatus of claim 5, wherein the second member extends coaxially through the first member.

12. The delivery apparatus of claim 11, wherein the threads of the first member are internal threads disposed on a socket of the of the distal end portion of the first member that are configured to mate with external threads on a threaded portion of the head of the actuator member.

13. The delivery apparatus of claim 11, wherein the distal end portion of the second member is an actuation head having a cross-section shaped to fit within a complementary shaped socket of the head of the actuator member.

14. The delivery apparatus of claim 13, wherein the cross-section of the actuation head of the second member is square-shaped.

15. A system comprising:
a prosthetic valve comprising a mechanically actuatable frame, the mechanically actuatable frame comprising at least one actuator member, the actuator member configured to be rotated, via rotation of a head thereof, to radially expand or compress the frame; and
a delivery apparatus comprising at least one actuation assembly, the actuation assembly comprising:
a first member comprising a threaded socket at its distal end that is configured to engage with external threads on a threaded portion of the head of the actuator member; and
a second member disposed coaxially within the first member, the second member comprising an actuation head at its distal end that is shaped to mate with and fit within a socket of the head of the actuator member, wherein the first member and the second member are rotatable together to rotate the actuator member and expand the prosthetic valve.

16. The system of claim 15, wherein the actuation assembly is movable between an engaged position and disengaged position with the frame, wherein in the engaged position the threaded socket of the first member is engaged with the external threads on the threaded portion of the head of the actuator member and the actuation head of the second member is disposed within and engaged with the socket of the head of the actuator member, and wherein in the engaged position the first member and the second member are rotatable together to rotate the actuator member and radially expand or compress the frame.

17. The system of claim 15, wherein the socket of the head of the actuator member extends through the threaded portion of the head of the actuator member such that the threads of the threaded portion are external to the socket of the head of the actuator.

18. The system of claim 15, wherein the actuation head has a cross-sectional shape that is complementary to a cross-sectional shape of the socket of the head of the actuator member.

19. The system of claim 15, wherein the actuation assembly further comprises an outer, third member comprising a pair of support extensions configured to engage with a post of the frame.

20. The system of claim 19, wherein the first member and the second member are rotatable relative to the third member.
